(19) United States
(12) Patent Application Publication
(10) Pub. No.: US 2025/0059275 A1
(43) Pub. Date: Feb. 20, 2025

(54) TIGIT ANTIBODIES AND USES THEREOF

(71) Applicant: SHANGHAI NIGENE BIOLOGICAL SCIENCE AND TECHNOLOGY CO., LTD., Shanghai (CN)

(72) Inventors: Xu Gao, Harbin (CN); Mistuo Oshimura, Tottori (JP); Zhimin Du, Harbin (CN); Kei Hiramatsu, Tottori (JP)

(21) Appl. No.: 18/721,114

(22) PCT Filed: Dec. 15, 2022
(86) PCT No.: PCT/CN2022/139205

§ 371 Date: Jun. 17, 2024

(30) Foreign Application Priority Data
Dec. 17, 2021 (WO) PCT/CN2021/139122

(51) Int. Cl. C07K 16/28; A61K 39/00; A61K 45/06; A61P 35/00

(52) U.S. Cl. CPC C07K 16/2803; A61K 45/06; A61P 35/00; A61K 2039/505; C07K 2317/565; C07K 2317/92

(57) ABSTRACT

Provided is an anti-TIGIT antibody or antigen-binding fragment thereof that specifically binds to TIGIT, a composition comprising the anti-TIGIT antibody or antigen-binding fragment thereof, and its use in the diagnosis and treatment of TIGIT-related diseases.

### CROSS REFERENCE TO RELATED APPLICATIONS

This application claims the priority of PCT Application No. PCT/CN2021/139122, filed on Dec. 17, 2021, and titled with “TIGIT ANTIBODIES AND USES THEREOF”, and the disclosures of which are hereby incorporated by reference.

### FIELD

The present invention relates to an antibody or antigen-binding fragment thereof that specifically binds to TIGIT (T cell immunoreceptor with Ig and ITIM domains), a pharmaceutical composition comprising the anti-TIGIT antibody or antigen-binding fragment thereof, and uses thereof.

### BACKGROUND OF INVENTION

In recent years, tumor immunotherapy has made a great breakthrough and has become a new hope for tumor therapy. In particular, the treatment scheme of blocking tumor immunosuppressive checkpoints represented by PD-1/PD-L1 and CTLA-4 has attracted much attention. Since 2000, FDA has successively approved the application of PD-1/PD-L1 monoclonal antibody in the clinical treatment of malignant tumors such as melanoma, non-small cell lung cancer and prostate cancer, and achieved good therapeutic results. However, the clinical response rate of PD-1/PD-L1 monoclonal antibody treatment is still limited, which limits its clinical application to a great extent. Therefore, finding new immunosuppressive checkpoints have become a research hotspot.

TIGIT is a new immunosuppressive factor discovered by Genentech team in 2009 (Nat Immunol, 2009, 10: 48-57). It is a member of PVR like protein family. TIGIT is expressed in T cells and NK cells, including CD4⁺ T cells, CD8⁺ T cells and Treg cells. Under normal conditions, the expression level of TIGIT is low, but when T cells and NK cells are activated, the expression of TIGIT increases significantly (J Immunol, 2012, 188:3869-3875, Cancer Cell 26, 923-937, Nat Immunol, 19, 723-732). At present, TIGIT ligands have been found including CD155, CD112 and CD113, and among them, the main ligand of TIGIT is CD155. Crystal structure analysis showed that TIGIT and CD155 form homodimers respectively, which further form heterotetramer by interaction between the ligands and receptors (Proc Natl Acad Sci USA 2012; 109: 5399-404). The binding affinity of TIGIT to CD112 or CD113 was significantly lower than that to CD155. CD155 is mainly expressed in dendritic cells, T cells, B cells and macrophages, as well as in non-hematopoietic tissues, such as kidney, nervous system and small intestine. Similar to TIGIT, activated receptors DNAM-1 and CD96 can also bind to CD155, but their affinity is weaker than TIGIT. In conclusion, the ligand-receptors action mode of TIGIT/CD155 is similar to CTLA-4/CD28 pathway. Inhibitory receptors with high affinity and activated receptors with low affinity compete to bind the same ligand, so as to accurately regulate the immune response. TIGIT combined with CD155 can play an immunosuppressive role by regulating DC function, inhibiting effector T cell activity, interfering with DNAM-1 coactivation and increasing Treg inhibition (Clinical and Experimental Immunology, 2020 May; 200 (2):108-119, Immunity 40, 569-581).

Several human-and mouse-based studies have shown that TIGIT is highly expressed in tumor infiltrating lymphocytes. TIGIT is upregulated in many malignant tumors, including melanoma, breast cancer, non-small cell lung cancer, colon adenocarcinoma, gastric cancer, acute myeloid leukemia and multiple myeloma (Clinical and Experimental Immunology, 2020 May; 200 (2): 108-119). Some studies also found that TIGIT was highly expressed in CD8+ T cells, tumor infiltrating Tregs and NK cells. In tumor patients, the expression of TIGIT in tumor infiltrating CD8⁺ T cells and NK cells is often consistent with the high expression of other inhibitory receptors such as PD-1, LAG-3, Tim-3 and the low expression of DNAM-1. High expression of TIGIT is often associated with poor prognosis of malignant tumors. The high expression of TIGIT in NK cells is related to the severity of the disease. The tumor growth of TIGIT knockout mice decreased significantly and the survival rate increased.

Because of its macromolecular properties, antibody drugs are often accompanied by immune related adverse events (irAEs). In TIGIT knockout mice, there were no spontaneous autoimmune symptoms and no hematopoietic cell development disorders. The occurrence of autoimmune diseases was increased only after hybridization with mice with autoimmune disease tendency. Compared with PD-1 and CTLA-4 mAbs, animal experiments showed that the incidence of irAEs was lower during the administration of anti-TIGIT mAbs (Oncoimmunology 2018; 7: e1445949). Therefore, the risk of side effects of antibody drugs targeting TIGIT is relatively low, and it is a high-quality candidate target for clinical anticancer drugs.

Published results of clinical trials showed that the combination of TIGIT antibody and PD-1/PD-L1 monoclonal antibody can significantly improve the response rate of patients, improve the treatment effect and solve the drug resistance of some patients (Cancers 2019; 11:877, Cancer Discov, 10: 1086-1087 (2020)). At present, no monoclonal antibody against TIGIT has been approved to market all over the world, so it is necessary to develop antibodies with high affinity and activity as candidate drugs.

### SUMMARY OF INVENTION

After a large number of experiments, the inventors of the present invention used a trans-chromosomal mouse (TC mAbIM mice) platform with all human antibody gene sequences to screen and unexpectedly obtain antibodies that specifically bind to TIGIT, which show an excellent affinity for TIGIT and have potential drug development prospects.

The present invention provides improved medicines and treatment methods for cancer and chronic viral infection comprising an anti-TIGIT antibody or antigen-binding fragment thereof that specifically binds to human TIGIT (huTIGIT). Provided herein are isolated antibodies, such as monoclonal antibodies, in particular human monoclonal antibodies, that specifically bind huTIGIT and have desirable functional properties, such as high-affinity specific binding to huTIGIT, binding to monkey TIGIT (e.g., cynomolgus TIGIT), the ability to block binding of TIGIT to PVR and Nectin-2, the ability to block the interaction of TIGIT with DNAM, or any combination of these properties.

The present invention relates to antibodies that compete with the antibodies having heavy and light chain variable domain sequences disclosed herein for binding to huTIGIT and that cross-block the antibodies having heavy and light chain variable domain sequences disclosed herein from binding huTIGIT.

In certain embodiments, the anti-TIGIT antibodies of the present invention, or antigen-binding fragments thereof, enhance an anti-tumor immune response, e.g., an antigen-specific T cell response. In other embodiments, the anti-TIGIT antibodies of the present invention, or antigen-binding fragments thereof, block TIGIT mediated inhibitory signaling allowing PVR/DNAM co-stimulation of NK cells to increase NK-mediated anti-tumor response killing. In yet another embodiment, the anti-TIGIT antibodies of the present invention, or antigen-binding fragments thereof, deplete a population of regulatory T cells within a tumor that would otherwise suppress the anti-tumor immune response. In yet another embodiment, anti-TIGIT antibodies of the present invention formatted as IgG1 deplete CD8+ exhausted T cells and Tregs, allowing for the influx of fresh, non-exhausted CD8+ T cells. In some embodiment, anti-TIGIT antibodies of the present invention formatted as IgG1 increase the proportion of CD8+TIL population in tumor microenvironment. In other embodiments, the anti-TIGIT antibodies of the present invention, or antigen-binding fragments thereof, act by one or more of the above-referenced mechanisms since the mechanisms are not necessarily mutually exclusive.

In certain embodiments, the anti-TIGIT antibodies of the present invention, or antigen-binding fragments thereof, do not bind to activating Fcγ receptors (FcγRs), e.g., in embodiments relying on enhancing the anti-tumor activity of TIGIT-expressing cells. In alternative embodiments, the anti-TIGIT antibodies of the present invention, or antigen-binding fragments thereof, bind to one or more activating FcγRs, e.g., in embodiments relying on the killing of TIGIT-expressing cells, such as exhausted CD8+ T cells or Tregs.

In the first aspect, the present invention provides an anti-TIGIT antibody or antigen-binding fragment thereof that specifically binds to TIGIT. The anti-TIGIT antibodies or antigen-binding fragments comprise a heavy chain variable region (VH) and/or a light chain variable region (VL), and wherein the heavy chain variable region comprises a CDRH1, a CDRH2, and a CDRH3, and the light chain variable region comprises a CDRL1, a CDRL2 and a CDRL3.

In some embodiments of the present invention, wherein
(a) the CDRH1 comprises a sequence of SEQ ID NO: 23, SEQ ID NO: 29, SEQ ID NO: 35, SEQ ID NO: 41, SEQ ID NO: 47, SEQ ID NO: 53, SEQ ID NO: 59, SEQ ID NO: 65, SEQ ID NO: 75 or SEQ ID NO: 81; or the CDRH1 comprises a sequence derived from SEQ ID NO: 23, SEQ ID NO: 29, SEQ ID NO: 35, SEQ ID NO: 41, SEQ ID NO: 47, SEQ ID NO: 53, SEQ ID NO: 59, SEQ ID NO: 65, SEQ ID NO: 75 or SEQ ID NO: 81 by addition, deletion or substitution of one or more amino acids;
(b) the CDRH2 comprises a sequence of SEQ ID NO: 24, SEQ ID NO: 30, SEQ ID NO: 36, SEQ ID NO: 42, SEQ ID NO: 48, SEQ ID NO: 54, SEQ ID NO: 60, SEQ ID NO: 66, SEQ ID NO: 76 or SEQ ID NO: 82; or the CDRH1 comprises a sequence derived from SEQ ID NO: 24, SEQ ID NO: 30, SEQ ID NO: 36, SEQ ID NO: 42, SEQ ID NO: 48, SEQ ID NO: 54, SEQ ID NO: 60, SEQ ID NO: 66, SEQ ID NO: 76 or SEQ ID NO: 82 by addition, deletion or substitution of one or more amino acids; and
(c) the CDRH3 comprises a sequence of SEQ ID NO: 25, SEQ ID NO: 31, SEQ ID NO: 37, SEQ ID NO: 43, SEQ ID NO: 49, SEQ ID NO: 55, SEQ ID NO: 61, SEQ ID NO: 67, SEQ ID NO: 77 or SEQ ID NO: 83; or the CDRH1 comprises a sequence derived from SEQ ID NO: 25, SEQ ID NO: 31, SEQ ID NO: 37, SEQ ID NO: 43, SEQ ID NO: 49, SEQ ID NO: 55, SEQ ID NO: 61, SEQ ID NO: 67, SEQ ID NO: 77 or SEQ ID NO: 83 by addition, deletion or substitution of one or more amino acids.

In some other embodiments of the present invention, wherein
(a) the CDRL1 comprises a sequence of SEQ ID NO: 20, SEQ ID NO: 26, SEQ ID NO: 32, SEQ ID NO: 38, SEQ ID NO: 44, SEQ ID NO: 50, SEQ ID NO: 56, SEQ ID NO: 62, SEQ ID NO: 72 or SEQ ID NO: 78; or the CDRH1 comprises a sequence derived from SEQ ID NO: 20, SEQ ID NO: 26, SEQ ID NO: 32, SEQ ID NO: 38, SEQ ID NO: 44, SEQ ID NO: 50, SEQ ID NO: 56, SEQ ID NO: 62, SEQ ID NO: 72 or SEQ ID NO: 78 by addition, deletion or substitution of one or more amino acids;
(b) the CDRL2 comprises a sequence of SEQ ID NO: 21, SEQ ID NO: 27, SEQ ID NO: 33, SEQ ID NO: 39, SEQ ID NO: 45, SEQ ID NO: 51, SEQ ID NO: 57, SEQ ID NO: 63, SEQ ID NO: 73 or SEQ ID NO: 79; or the CDRH1 comprises a sequence derived from SEQ ID NO: 21, SEQ ID NO: 27, SEQ ID NO: 33, SEQ ID NO: 39, SEQ ID NO: 45, SEQ ID NO: 51, SEQ ID NO: 57, SEQ ID NO: 63, SEQ ID NO: 73 or SEQ ID NO: 79 by addition, deletion or substitution of one or more amino acids; and
(c) the CDRL3 comprises a sequence of SEQ ID NO: 22, SEQ ID NO: 28, SEQ ID NO: 34, SEQ ID NO: 40, SEQ ID NO: 46, SEQ ID NO: 52, SEQ ID NO: 58, SEQ ID NO: 64, SEQ ID NO: 74 or SEQ ID NO: 80; or the CDRH1 comprises a sequence derived from SEQ ID NO: 22, SEQ ID NO: 28, SEQ ID NO: 34, SEQ ID NO: 40, SEQ ID NO: 46, SEQ ID NO: 52, SEQ ID NO: 58, SEQ ID NO: 64, SEQ ID NO: 74 or SEQ ID NO: 80 by addition, deletion or substitution of one or more amino acids.

In some other embodiments of the present invention, wherein
(a) the CDRH1 comprises a sequence having at least 80%, at least 85%, at least 90%, at least 95%, at least 96%, at least 97%, at least 98%, at least 99% identity to a sequence selected from the group consisting of SEQ ID NO: 23, SEQ ID NO: 29, SEQ ID NO: 35, SEQ ID NO: 41, SEQ ID NO: 47, SEQ ID NO: 53, SEQ ID NO: 59, SEQ ID NO: 65, SEQ ID NO: 75 and SEQ ID NO: 81;
(b) the CDRH2 comprises a sequence having at least 80%, at least 85%, at least 90%, at least 95%, at least 96%, at least 97%, at least 98%, at least 99% identity to a sequence selected from the group consisting of SEQ ID NO: 24, SEQ ID NO: 30, SEQ ID NO: 36, SEQ ID NO: 42, SEQ ID NO: 48, SEQ ID NO: 54, SEQ ID NO: 60, SEQ ID NO: 66, SEQ ID NO: 76 and SEQ ID NO: 82; and
(c) the CDRH3 comprises a sequence having at least 80%, at least 85%, at least 90%, at least 95%, at least 96%, at least 97%, at least 98%, at least 99% identity to a sequence selected from the group consisting of SEQ ID NO: 25, SEQ ID NO: 31, SEQ ID NO: 37, SEQ ID NO: 43, SEQ ID NO: 49, SEQ ID NO: 55, SEQ ID NO: 61, SEQ ID NO: 67, SEQ ID NO: 77 and SEQ ID NO: 83; and/or
(d) the CDRL1 comprises a sequence having at least 80%, at least 85%, at least 90%, at least 95%, at least 96%, at least 97%, at least 98%, at least 99% identity to a sequence selected from the group consisting of SEQ ID NO: 20, SEQ ID NO: 26, SEQ ID NO: 32, SEQ ID NO: 38, SEQ ID NO: 44, SEQ ID NO: 50, SEQ ID NO: 56, SEQ ID NO: 62, SEQ ID NO: 72 and SEQ ID NO: 78;
(e) the CDRL2 comprises a sequence having at least 80%, at least 85%, at least 90%, at least 95%, at least 96%, at least 97%, at least 98%, at least 99% identity to a sequence selected from the group consisting of SEQ ID NO: 21, SEQ ID NO: 27, SEQ ID NO: 33, SEQ ID NO: 39, SEQ ID NO: 45, SEQ ID NO: 51, SEQ ID NO: 57, SEQ ID NO: 63, SEQ ID NO: 73 and SEQ ID NO: 79; and
(f) the CDRL3 comprises a sequence having at least 80%, at least 85%, at least 90%, at least 95%, at least 96%, at least 97%, at least 98%, at least 99% identity to a sequence selected from the group consisting of SEQ ID NO: 22, SEQ ID NO: 28, SEQ ID NO: 34, SEQ ID NO: 40, SEQ ID NO: 46, SEQ ID NO: 52, SEQ ID NO: 58, SEQ ID NO: 64, SEQ ID NO: 74 and SEQ ID NO: 80.

In some other embodiments of the present invention, wherein
(a) the CDRH1 comprises a sequence selected from the group consisting of SEQ ID NO: 23, SEQ ID NO: 29, SEQ ID NO: 35, SEQ ID NO: 41, SEQ ID NO: 47, SEQ ID NO: 53, SEQ ID NO: 59, SEQ ID NO: 65, SEQ ID NO: 75 and SEQ ID NO: 81;
(b) the CDRH2 comprises a sequence selected from the group consisting of SEQ ID NO: 24, SEQ ID NO: 30, SEQ ID NO: 36, SEQ ID NO: 42, SEQ ID NO: 48, SEQ ID NO: 54, SEQ ID NO: 60, SEQ ID NO: 66, SEQ ID NO: 76 and SEQ ID NO: 82;
(c) the CDRH3 comprises a sequence selected from the group consisting of SEQ ID NO: 25, SEQ ID NO: 31, SEQ ID NO: 37, SEQ ID NO: 43, SEQ ID NO: 49, SEQ ID NO: 55, SEQ ID NO: 61, SEQ ID NO: 67, SEQ ID NO: 77 and SEQ ID NO: 83;
(d) the CDRL1 comprises a sequence selected from the group consisting of SEQ ID NO: 20, SEQ ID NO: 26, SEQ ID NO: 32, SEQ ID NO: 38, SEQ ID NO: 44, SEQ ID NO: 50, SEQ ID NO: 56, SEQ ID NO: 62, SEQ ID NO: 72 and SEQ ID NO: 78;
(e) the CDRL2 comprises a sequence selected from the group consisting of SEQ ID NO: 21, SEQ ID NO: 27, SEQ ID NO: 33, SEQ ID NO: 39, SEQ ID NO: 45, SEQ ID NO: 51, SEQ ID NO: 57, SEQ ID NO: 63, SEQ ID NO: 73 and SEQ ID NO: 79; and
(f) the CDRL3 comprises a sequence selected from the group consisting of SEQ ID NO: 22, SEQ ID NO: 28, SEQ ID NO: 34, SEQ ID NO: 40, SEQ ID NO: 46, SEQ ID NO: 52, SEQ ID NO: 58, SEQ ID NO: 64, SEQ ID NO: 74 and SEQ ID NO: 80.

In some other embodiments of the present invention, wherein the heavy chain variable region comprises a CDRH1, a CDRH2 and a CDRH3, and wherein
(a) the CDRH1 comprises a sequence as shown in SEQ ID NO: 23, the CDRH2 comprises a sequence as shown in SEQ ID NO: 24 and the CDRH3 comprises a sequence as shown in SEQ ID NO: 25;
(b) the CDRH1 comprises a sequence as shown in SEQ ID NO: 29, the CDRH2 comprises a sequence as shown in SEQ ID NO: 30 and the CDRH3 comprises a sequence as shown in SEQ ID NO: 31;
(c) the CDRH1 comprises a sequence as shown in SEQ ID NO: 35, the CDRH2 comprises a sequence as shown in SEQ ID NO: 36 and the CDRH3 comprises a sequence as shown in SEQ ID NO: 37;
(d) the CDRH1 comprises a sequence as shown in SEQ ID NO: 41, the CDRH2 comprises a sequence as shown in SEQ ID NO: 42 and the CDRH3 comprises a sequence as shown in SEQ ID NO: 43;
(e) the CDRH1 comprises a sequence as shown in SEQ ID NO: 47, the CDRH2 comprises a sequence as shown in SEQ ID NO: 48 and the CDRH3 comprises a sequence as shown in SEQ ID NO: 49;
(f) the CDRH1 comprises a sequence as shown in SEQ ID NO: 53, the CDRH2 comprises a sequence as shown in SEQ ID NO: 54 and the CDRH3 comprises a sequence as shown in SEQ ID NO: 55;
(g) the CDRH1 comprises a sequence as shown in SEQ ID NO: 59, the CDRH2 comprises a sequence as shown in SEQ ID NO: 60 and the CDRH3 comprises a sequence as shown in SEQ ID NO: 61;
(h) the CDRH1 comprises a sequence as shown in SEQ ID NO: 65, the CDRH2 comprises a sequence as shown in SEQ ID NO: 66 and the CDRH3 comprises a sequence as shown in SEQ ID NO: 67;
(i) the CDRH1 comprises a sequence of SEQ ID NO: 75, the CDRH2 comprises a sequence of SEQ ID NO: 76 and the CDRH3 comprises a sequence of SEQ ID NO: 77; or
(j) the CDRH1 comprises a sequence of SEQ ID NO: 81, the CDRH2 comprises a sequence of SEQ ID NO: 82 and the CDRH3 comprises a sequence of SEQ ID NO: 83.

In some other embodiments of the present invention, wherein the light chain variable region comprises a CDRL1, a CDRL2 and a CDRL3, and wherein
(a) the CDRL1 comprises a sequence as shown in SEQ ID NO: 20, the CDRL2 comprises a sequence as shown in SEQ ID NO: 21 and the CDRL3 comprises a sequence as shown in SEQ ID NO: 22;
(b) the CDRL1 comprises a sequence as shown in SEQ ID NO: 26, the CDRL2 comprises a sequence as shown in SEQ ID NO: 27 and the CDRL3 comprises a sequence as shown in SEQ ID NO: 28;
(c) the CDRL1 comprises a sequence as shown in SEQ ID NO: 32, the CDRL2 comprises a sequence as shown in SEQ ID NO: 33 and the CDRL3 comprises a sequence as shown in SEQ ID NO: 34;
(d) the CDRL1 comprises a sequence as shown in SEQ ID NO: 38, the CDRL2 comprises a sequence as shown in SEQ ID NO: 39 and the CDRL3 comprises a sequence as shown in SEQ ID NO: 40;
(e) the CDRL1 comprises a sequence as shown in SEQ ID NO: 44, the CDRL2 comprises a sequence as shown in SEQ ID NO: 45 and the CDRL3 comprises a sequence as shown in SEQ ID NO: 46;
(f) the CDRL1 comprises a sequence as shown in SEQ ID NO: 50, the CDRL2 comprises a sequence as shown in SEQ ID NO: 51 and the CDRL3 comprises a sequence as shown in SEQ ID NO: 52;
(g) the CDRL1 comprises a sequence as shown in SEQ ID NO: 56, the CDRL2 comprises a sequence as shown in SEQ ID NO: 57 and the CDRL3 comprises a sequence as shown in SEQ ID NO: 58;
(h) the CDRL1 comprises a sequence as shown in SEQ ID NO: 62, the CDRL2 comprises a sequence as shown in SEQ ID NO: 63 and the CDRL3 comprises a sequence as shown in SEQ ID NO: 64;
(i) the CDRL1 comprises a sequence of SEQ ID NO: 72, the CDRL2 comprises a sequence of SEQ ID NO: 73 and the CDRL3 comprises a sequence of SEQ ID NO: 74; or
(j) the CDRL1 comprises a sequence of SEQ ID NO: 78, the CDRL2 comprises a sequence of SEQ ID NO: 79 and the CDRL3 comprises a sequence of SEQ ID NO: 80.

In some other embodiments of the present invention, wherein
(a) the CDRH1 comprises a sequence as shown in SEQ ID NO: 23, the CDRH2 comprises a sequence as shown in SEQ ID NO: 24 and the CDRH3 comprises a sequence as shown in SEQ ID NO: 25; and the CDRL1 comprises a sequence as shown in SEQ ID NO: 20, the CDRL2 comprises a sequence as shown in SEQ ID NO: 21 and the CDRL3 comprises a sequence as shown in SEQ ID NO: 22;
(b) the CDRH1 comprises a sequence as shown in SEQ ID NO: 29, the CDRH2 comprises a sequence as shown in SEQ ID NO: 30 and the CDRH3 comprises a sequence as shown in SEQ ID NO: 31; and the CDRL1 comprises a sequence as shown in SEQ ID NO: 26, the CDRL2 comprises a sequence as shown in SEQ ID NO: 27 and the CDRL3 comprises a sequence as shown in SEQ ID NO: 28;
(c) the CDRH1 comprises a sequence as shown in SEQ ID NO: 35, the CDRH2 comprises a sequence as shown in SEQ ID NO: 36 and the CDRH3 comprises a sequence as shown in SEQ ID NO: 37; and the CDRL1 comprises a sequence as shown in SEQ ID NO: 32, the CDRL2 comprises a sequence as shown in SEQ ID NO: 33 and the CDRL3 comprises a sequence as shown in SEQ ID NO: 34;
(d) the CDRH1 comprises a sequence as shown in SEQ ID NO: 41, the CDRH2 comprises a sequence as shown in SEQ ID NO: 42 and the CDRH3 comprises a sequence as shown in SEQ ID NO: 43; and the CDRL1 comprises a sequence as shown in SEQ ID NO: 38, the CDRL2 comprises a sequence as shown in SEQ ID NO: 39 and the CDRL3 comprises a sequence as shown in SEQ ID NO: 40;
(e) the CDRH1 comprises a sequence as shown in SEQ ID NO: 47, the CDRH2 comprises a sequence as shown in SEQ ID NO: 48 and the CDRH3 comprises a sequence as shown in SEQ ID NO: 49; and the CDRL1 comprises a sequence as shown in SEQ ID NO: 44, the CDRL2 comprises a sequence as shown in SEQ ID NO: 45 and the CDRL3 comprises a sequence as shown in SEQ ID NO: 46;
(f) the CDRH1 comprises a sequence as shown in SEQ ID NO: 53, the CDRH2 comprises a sequence as shown in SEQ ID NO: 54 and the CDRH3 comprises a sequence as shown in SEQ ID NO: 55; and the CDRL1 comprises a sequence as shown in SEQ ID NO: 50, the CDRL2 comprises a sequence as shown in SEQ ID NO: 51 and the CDRL3 comprises a sequence as shown in SEQ ID NO: 52;
(g) the CDRH1 comprises a sequence as shown in SEQ ID NO: 59, the CDRH2 comprises a sequence as shown in SEQ ID NO: 60 and the CDRH3 comprises a sequence as shown in SEQ ID NO: 61; and the CDRL1 comprises a sequence as shown in SEQ ID NO: 56, the CDRL2 comprises a sequence as shown in SEQ ID NO: 57 and the CDRL3 comprises a sequence as shown in SEQ ID NO: 58;
(h) the CDRH1 comprises a sequence as shown in SEQ ID NO: 65, the CDRH2 comprises a sequence as shown in SEQ ID NO: 66 and the CDRH3 comprises a sequence as shown in SEQ ID NO: 67; and the CDRL1 comprises a sequence as shown in SEQ ID NO: 62, the CDRL2 comprises a sequence as shown in SEQ ID NO: 63 and the CDRL3 comprises a sequence as shown in SEQ ID NO: 64;
(i) the CDRH1 comprises a sequence of SEQ ID NO: 75, the CDRH2 comprises a sequence of SEQ ID NO: 76 and the CDRH3 comprises a sequence of SEQ ID NO: 77; and the CDRL1 comprises a sequence of SEQ ID NO: 72, the CDRL2 comprises a sequence of SEQ ID NO: 73 and the CDRL3 comprises a sequence of SEQ ID NO: 74; or
(j) the CDRH1 comprises a sequence of SEQ ID NO: 81, the CDRH2 comprises a sequence of SEQ ID NO: 82 and the CDRH3 comprises a sequence of SEQ ID NO: 83; and the CDRL1 comprises a sequence of SEQ ID NO: 78, the CDRL2 comprises a sequence of SEQ ID NO: 79 and the CDRL3 comprises a sequence of SEQ ID NO: 80.

In some other embodiments of the present invention, wherein
(a) the sequence of CDRH1 is shown in SEQ ID NO: 23, the sequence of CDRH2 is shown in SEQ ID NO: 24 and the sequence of CDRH3 is shown in SEQ ID NO: 25; and the sequence of CDRL1 is shown in SEQ ID NO: 20, the sequence of CDRL2 is shown in SEQ ID NO: 21 and the sequence of CDRL3 is shown in SEQ ID NO: 22;
(b) the sequence of CDRH1 is shown in SEQ ID NO: 29, the sequence of CDRH2 is shown in SEQ ID NO: 30 and the sequence of CDRH3 is shown in SEQ ID NO: 31; and the sequence of CDRL1 is shown in SEQ ID NO: 26, the sequence of CDRL2 is shown in SEQ ID NO: 27 and the sequence of CDRL3 is shown in SEQ ID NO: 28;
(c) the sequence of CDRH1 is shown in SEQ ID NO: 35, the sequence of CDRH2 is shown in SEQ ID NO: 36 and the sequence of CDRH3 is shown in SEQ ID NO: 37; and the sequence of CDRL1 is shown in SEQ ID NO: 32, the sequence of CDRL2 is shown in SEQ ID NO: 33 and the sequence of CDRL3 is shown in SEQ ID NO: 34;
(d) the sequence of CDRH1 is shown in SEQ ID NO: 41, the sequence of CDRH2 is shown in SEQ ID NO: 42 and the sequence of CDRH3 is shown in SEQ ID NO: 43; and the sequence of CDRL1 is shown in SEQ ID NO: 38, the sequence of CDRL2 is shown in SEQ ID NO: 39 and the sequence of CDRL3 is shown in SEQ ID NO: 40;
(e) the sequence of CDRH1 is shown in SEQ ID NO: 47, the sequence of CDRH2 is shown in SEQ ID NO: 48 and the sequence of CDRH3 is shown in SEQ ID NO: 49; and the sequence of CDRL1 is shown in SEQ ID NO: 44, the sequence of CDRL2 is shown in SEQ ID NO: 45 and the sequence of CDRL3 is shown in SEQ ID NO: 46;
(f) the sequence of CDRH1 is shown in SEQ ID NO: 53, the sequence of CDRH2 is shown in SEQ ID NO: 54 and the sequence of CDRH3 is shown in SEQ ID NO: 55; and the sequence of CDRL1 is shown in SEQ ID NO: 50, the sequence of CDRL2 is shown in SEQ ID NO: 51 and the sequence of CDRL3 is shown in SEQ ID NO: 52;
(g) the sequence of CDRH1 is shown in SEQ ID NO: 59, the sequence of CDRH2 is shown in SEQ ID NO: 60 and the sequence of CDRH3 is shown in SEQ ID NO: 61; and the sequence of CDRL1 is shown in SEQ ID NO: 56, the sequence of CDRL2 is shown in SEQ ID NO: 57 and the sequence of CDRL3 is shown in SEQ ID NO: 58;
(h) the sequence of CDRH1 is shown in SEQ ID NO: 65, the sequence of CDRH2 is shown in SEQ ID NO: 66 and the sequence of CDRH3 is shown in SEQ ID NO: 67; and the sequence of CDRL1 is shown in SEQ ID NO: 62, the sequence of CDRL2 is shown in SEQ ID NO: 63 and the sequence of CDRL3 is shown in SEQ ID NO: 64;
(i) the sequence of CDRH1 is shown in SEQ ID NO: 75, the sequence of CDRH2 is shown in SEQ ID NO: 76 and the sequence of CDRH3 is shown in SEQ ID NO: 77; and the sequence of CDRL1 is shown in SEQ ID NO: 72, the sequence of CDRL2 is shown in SEQ ID NO: 73 and the sequence of CDRL3 is shown in SEQ ID NO: 74; or
(j) the sequence of CDRH1 is shown in SEQ ID NO: 81, the sequence of CDRH2 is shown in SEQ ID NO: 82 and the sequence of CDRH3 is shown in SEQ ID NO: 83; and the sequence of CDRL1 is shown in SEQ ID NO: 78, the sequence of CDRL2 is shown in SEQ ID NO: 79 and the sequence of CDRL3 is shown in SEQ ID NO: 80.

In some other embodiments of the present invention, wherein the heavy chain variable region comprises a sequence selected from the group consisting of SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 69 and SEQ ID NO: 71.

In some other embodiments of the present invention, wherein the light chain variable region comprises a sequence selected from the group consisting of SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 68 and SEQ ID NO: 70.

In some other embodiments of the present invention, wherein
(a) the heavy chain variable region comprises a sequence shown in SEQ ID NO: 5 and the light chain variable region comprises a sequence shown in SEQ ID NO: 4;
(b) the heavy chain variable region comprises a sequence shown in SEQ ID NO: 7 and the light chain variable region comprises a sequence shown in SEQ ID NO: 6;
(c) the heavy chain variable region comprises a sequence shown in SEQ ID NO: 9 and the light chain variable region comprises a sequence shown in SEQ ID NO: 8;
(d) the heavy chain variable region comprises a sequence shown in SEQ ID NO: 11 and the light chain variable region comprises a sequence shown in SEQ ID NO: 10;
(e) the heavy chain variable region comprises a sequence shown in SEQ ID NO: 13 and the light chain variable region comprises a sequence shown in SEQ ID NO: 12;
(f) the heavy chain variable region comprises a sequence shown in SEQ ID NO: 15 and the light chain variable region comprises a sequence shown in SEQ ID NO: 14;
(g) the heavy chain variable region comprises a sequence shown in SEQ ID NO: 17 and the light chain variable region comprises a sequence shown in SEQ ID NO: 16; or
(h) the heavy chain variable region comprises a sequence shown in SEQ ID NO: 19 and the light chain variable region comprises a sequence shown in SEQ ID NO: 18.

In some other embodiments of the present invention, wherein
(a) the sequence of the heavy chain variable region is shown in SEQ ID NO: 5 and the sequence of the light chain variable region is shown in SEQ ID NO: 4;
(b) the sequence of the heavy chain variable region is shown in SEQ ID NO: 7 and the sequence of the light chain variable region is shown in SEQ ID NO: 6;
(c) the sequence of the heavy chain variable region is shown in SEQ ID NO: 9 and the sequence of the light chain variable region is shown in SEQ ID NO: 8;
(d) the sequence of the heavy chain variable region is shown in SEQ ID NO: 11 and the sequence of the light chain variable region is shown in SEQ ID NO: 10;
(e) the sequence of the heavy chain variable region is shown in SEQ ID NO: 13 and the sequence of the light chain variable region is shown in SEQ ID NO: 12;
(f) the sequence of the heavy chain variable region is shown in SEQ ID NO: 15 and the sequence of the light chain variable region is shown in SEQ ID NO: 14;
(g) the sequence of the heavy chain variable region is shown in SEQ ID NO: 17 and the sequence of the light chain variable region is shown in SEQ ID NO: 16;
(h) the sequence of the heavy chain variable region is shown in SEQ ID NO: 19 and the sequence of the light chain variable region is shown in SEQ ID NO: 18;
(i) the heavy chain variable region comprises a sequence of SEQ ID NO: 69 and the light chain variable region comprises a sequence of SEQ ID NO: 68; or
(j) the heavy chain variable region comprises a sequence of SEQ ID NO: 71 and the light chain variable region comprises a sequence of SEQ ID NO: 70.

In some other embodiments of the present invention, the anti-TIGIT antibody or antigen-binding fragment thereof comprises a heavy chain variable region (VH) and/or a light chain variable region (VL), and wherein
(a) the VH comprises a CDRH1, a CDRH2 and a CDRH3 of a VH shown in SEQ ID NO: 5, and/or the VL comprises a CDRL1, a CDRL2 and a CDRL3 of a VL shown in SEQ ID NO: 4;
(b) the VH comprises a CDRH1, a CDRH2 and a CDRH3 of a VH shown in SEQ ID NO: 7, and/or the VL comprises a CDRH1, a CDRL2 and a CDRL3 of a VL shown in SEQ ID NO: 6;
(c) the VH comprises a CDRH1, a CDRH2 and a CDRH3 of a VH shown in SEQ ID NO: 9, and/or the VL comprises a CDRH1, a CDRL2 and a CDRL3 of a VL shown in SEQ ID NO: 8;
(d) the VH comprises a CDRH1, a CDRH2 and a CDRH3 of a VH shown in SEQ ID NO: 11, and/or the VL comprises a CDRH1, a CDRL2 and a CDRL3 of a VL shown in SEQ ID NO: 10;
(e) the VH comprises a CDRH1, a CDRH2 and a CDRH3 of a VH shown in SEQ ID NO: 13, and/or the VL comprises a CDRH1, a CDRL2 and a CDRL3 of a VL shown in SEQ ID NO: 12;
(f) the VH comprises a CDRH1, a CDRH2 and a CDRH3 of a VH shown in SEQ ID NO: 15, and/or the VL comprises a CDRH1, a CDRL2 and a CDRL3 of a VL shown in SEQ ID NO: 14;
(g) the VH comprises a CDRH1, a CDRH2 and a CDRH3 of a VH shown in SEQ ID NO: 17, and/or the VL comprises a CDRH1, a CDRL2 and a CDRL3 of a VL shown in SEQ ID NO: 16;
(h) the VH comprises a CDRH1, a CDRH2 and a CDRH3 of a VH shown in SEQ ID NO: 19, and/or the VL comprises a CDRH1, a CDRL2 and a CDRL3 of a VL shown in SEQ ID NO: 18;
(i) the VH comprises a CDRH1, a CDRH2 and a CDRH3 of a VH set forth in SEQ ID NO: 69, and/or the VL comprises a CDRH1, a CDRL2 and a CDRL3 of a VL set forth in SEQ ID NO: 68; or
(j) the VH comprises a CDRH1, a CDRH2 and a CDRH3 of a VH set forth in SEQ ID NO: 71, and/or the VL comprises a CDRH1, a CDRL2 and a CDRL3 of a VL set forth in SEQ ID NO: 70.

In certain embodiments, the isolated monoclonal antibodies of the present invention, or antigen binding fragments thereof, (a) some antibody can block the binding of itself (1B2-8C) and Tiragolumab, 4A042-H3, 4A042-H7 and 4B030a, partially block the binding of 4A063, but not 4B037a, 4B056a, 4A063, 4D035a and 4E061a; (b) some antibody can block the binding of itself (4A042-H3), 1B2-8C, 4A042-H7 and 4B030a without blocking the binding of 4B037a, 4B056a, 4A063, 4D035a and 4E061a; (c) some antibody can block the binding of itself (4A042-H7), 1B2-8C, 4A042-H3 and 4B030a, partially block the binding of 4B037a and 4A063, but not 4B056a, 4D035a and 4E061a; (d) some antibody can block the binding of itself (4B030a), partially block the binding of 4B056a and 4A063, and do not block the binding of 4B037a, 4D035a and 4E061a; (e) some antibodies can block the binding of themselves (4B037a, 4A063), 4B056a, 4D035a and 4E061a, and partially block the binding of Tiragolumab, 1B2-8C, 4A042-H3, 4A042-H7 and 4B030a; (f) some antibody can block the binding of itself (4B056a),4B037a, 4A063, 4D035a and 4E061a, partially block the binding of Tiragolumab, 1B2-8C, 4A042-H3 and 4B030a, but not 4A042-H7; (g) some antibodies can block the binding of themselves (4D035a,4E061a), 4B037a, 4B056a, 4A063, without blocking the binding of Tiragolumab, 1B2-8C, 4A042-H3, 4A042-H7 and 4B030a.

In some embodiments, the anti-huTIGIT antibodies of the present invention, or antigen binding fragments thereof, also bind to cynomolgus TIGIT.

In some other embodiments of the present invention, the anti-TIGIT antibody or antigen-binding fragment thereof further comprises a heavy chain constant region selected from a heavy chain constant region of IgG1, IgG2, IgG3, IgG4, IgA, IgM, IgE and IgD. In some examples of the present invention, the heavy chain constant region is a heavy chain constant region of human Ig G or variants thereof.

In some other embodiments of the present invention, the anti-TIGIT antibody or antigen-binding fragment thereof is in a form selected from the group consisting of F(ab′)₂, Fab′, Fab, Fv, scFv, bispecific antibody, and a combination thereof.

The present invention also provides immunoconjugates comprising the anti-TIGIT antibodies described herein, linked to an agent, such as a detectable label or cytotoxic agent.

In other embodiments, the antigen-binding domains of the antibodies of the present invention are present in bispecific molecules further comprising an antigen-binding domain that binds specifically to a different immunomodulatory receptor, including but not limited to PD-1, CTLA-4 or LAG3.

In the second aspect, the present invention provides a polynucleotide encoding the anti-TIGIT antibodies, or antigen-binding fragment thereof.

In the third aspect, the present invention provides an expression vector expressing the anti-TIGIT antibodies or antigen-binding fragment thereof.

In the fourth aspect, the present invention provides an engineered cell comprising the vector expressing the anti-TIGIT antibody or antigen-binding fragment.

In the fifth aspect, the present invention provides a pharmaceutical composition comprising the anti-TIGIT antibody or antigen-binding fragment thereof of the first aspect, the polynucleotide of the second aspect, the vector of the third aspect, or the cells of the fourth aspect, and a pharmaceutically acceptable carrier. The present invention further provides an antibody-drug conjugate comprising the first aspect of the anti-TIGIT antibody or antigen-binding fragment. Also provided herein are kits containing the anti-TIGIT antibodies, or antigen-binding fragments thereof, and instructions for use.

In the sixth aspect, the present invention provides use of the anti-TIGIT antibody or antigen-binding fragment thereof of the first aspect, the polynucleotide of the second aspect, the vector of the third aspect, the cells of the fourth aspect, or the pharmaceutical composition of the fifth aspect in the manufacture of a medicament for treating TIGIT-related diseases. Preferably, the TIGIT-related disease is a T cell dysfunction disease; more preferably, the TIGIT-related disease is a tumor, an immune disease, or an infectious disease; more preferably, the cancer is selected from the group consisting of melanoma, breast cancer, non-small cell lung cancer, colon adenocarcinoma, gastric cancer, acute myeloid leukemia, and multiple myeloma. More preferably, cells of the tumor are CD155 positive or PVR positive.

In some embodiments, the present invention provides a method of enhancing an antigen-specific T cell response comprising contacting the T cell with an anti-huTIGIT antibody of the present invention, or antigen-binding fragment thereof, such that an antigen-specific T cell response is enhanced, e.g., by reduction of an inhibitory signal that would otherwise dampen the anti-tumor response. In some embodiments, the antigen-specific T cell is a tumor-antigen specific effector T cell, such as a CD8+ T cell, and the enhancement, e.g., through blocking of a TIGIT-mediated inhibitory effect, results in increased anti-tumor activity. Anti-huTIGIT antibodies of the present invention, or antigen-binding fragments thereof, may also reduce inhibitory signals in NK cells and thus increase their anti-tumor activity. Without intending to be limited by theory, anti-huTIGIT antibodies of the present invention increase effector T cell or NK cell function by blocking the binding of TIGIT to PVR, thus reducing or eliminating an inhibitory signal that would otherwise be delivered to the cell. Alternatively, or in addition, anti-TIGIT antibodies of the present invention, or antigen-binding fragments thereof, may inhibit the interaction between TIGIT and DNAM-1/CD226 that would otherwise reduce DNAM-1-mediated immune activation.

The present invention provides a method of reducing or depleting Tregs in a tumor in a subject in need thereof comprising administering an effective amount of an anti-huTIGIT antibody of the present invention, wherein the antibody has effector function or enhanced effector function, to reduce the number of Tregs in the tumor.

The present invention provides a method of enhancing an immune response in a subject comprising administering an effective amount of an anti-huTIGIT antibody of the present invention, or antigen-binding fragment thereof, to the subject such that an immune response in the subject is enhanced. In certain embodiments, the subject has a tumor, and an immune response against the tumor is enhanced. In another embodiment, the subject has a viral infection, and an anti-viral immune response is enhanced.

The present invention also provides a method of inhibiting the growth of tumors in a subject comprising administering to the subject an anti-huTIGIT antibody of the present invention, or antigen-binding fragment thereof, such that growth of the tumor is inhibited.

The present invention further provides a method of treating cancer, e.g., by immunotherapy, comprising administering to a subject in need thereof a therapeutically effective amount an anti-huTIGIT antibody of the present invention, or antigen-binding fragment thereof, e.g., as a pharmaceutical composition, thereby treating cancer. In certain embodiments, the cancer is bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, and virus-related cancer. In certain embodiments, the cancer is metastatic cancer, refractory cancer, or recurrent cancer.

In the seventh aspect, the present invention provides a method of treating TIGIT-related diseases comprising administering to the subject in need thereof an effective amount of the anti-TIGIT antibody or antigen-binding fragment thereof of the first aspect, the polynucleotide of the second aspect, the vector of the third aspect, the cells of the fourth aspect, or the pharmaceutical composition of the fifth aspect.

In the eighth aspect, the present invention provides a pharmaceutical composition for use in treating TIGIT-related diseases, wherein the pharmaceutical composition comprises the anti-TIGIT antibody or antigen-binding fragment thereof of the first aspect, the polynucleotide of the second aspect, the vector of the third aspect, or the cells of the fourth aspect. In combination with, or as a bispecific reagent with, one or more additional therapeutics, for example, an anti-PD-1 antibody, an anti-PD-L1 antibody, an anti-LAG3 antibody, an anti-GITR antibody, an anti-OX40 antibody, an anti-CD73 antibody, an anti-CD40 antibody, an anti-CD137 mAb, an anti-CD27 mAb, an anti-CSF-1R antibody, and/or an anti-CTLA-4 antibody, a TLR agonist, or a small molecule antagonist of IDO or TGFβ. In specific embodiments, anti-huTIGIT therapy is combined with anti-PD-1 and/or anti-PD-L1 therapy, e.g., treatment with an antibody or antigen-binding fragment thereof that binds to human PD-1 or an antibody or antigen-binding fragment thereof that binds to human PD-L1.

The present invention also provides methods of detecting the presence of TIGIT in a sample, on a cell within a sample (e.g., FACS), or in specific locations in a cell or tissue (e.g., IHC), or of sorting cells based on the presence or absence of TIGIT on their surface (e.g., FACS), comprising contacting the sample with an anti-huTIGIT antibody of the present invention, or an antigen-binding fragment thereof, under conditions that allow for the formation of a complex between the antibody, or antigen-binding fragment thereof, and TIGIT, and detecting the formation of the complex. In some embodiments, the anti-TIGIT antibody used for detection is conjugated with a detectable label.

The present invention uses a trans-chromosomal mouse (TC-mAb™ mice) model into which fully human antibody gene sequences (including gene regulatory sequences) are transferred. The target antigen is used to immunize the trans-chromosomal mice to obtain fully humanized antibodies directly. The obtained antibodies do not require subsequent humanization and affinity modification, reducing costs and shortening the development cycle. In addition, the antibodies are fully human antibodies derived from trans-chromosomal mouse, which significantly reduces its immunogenicity and is more conducive to drug development.

### BRIEF DESCRIPTION OF DRAWINGS

FIG. 1 is a graph showing the results of huTIGIT binding affinity using recombinant huTIGIT protein and indicated TC-mAb™ mice-derived anti-TIGIT antibodies in the present invention. See Example 3.

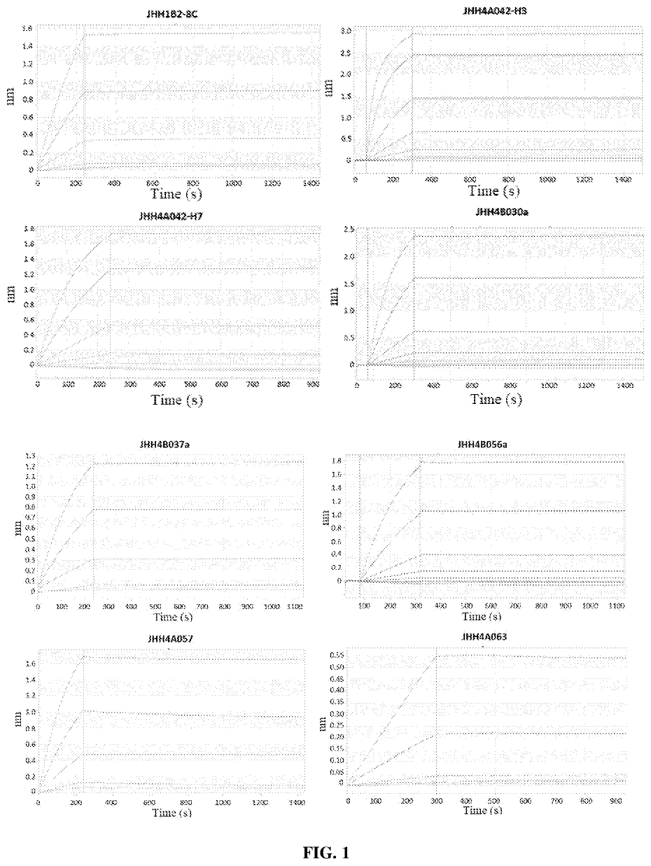

FIG. 2A, 2B and 2C show the results of epitope competition using recombinant huTIGIT protein and indicated TC-mAb™ mice-derived anti-TIGIT antibodies in the present invention. See Example 3.

FIG. 3A is a graph showing the results of CHO-TIGIT binding assay using human TIGIT-expressing CHO cells and the indicated TC-mAb™ mice-derived anti-TIGIT antibodies in the present invention at varying concentrations.

FIG. 3B is a graph showing the results of CHO-TIGIT binding assay using cynomolgus monkey TIGIT (mkTIGIT)-expressing CHO cells and the indicated TC-mAb™ mice-derived anti-TIGIT antibodies in the present invention at varying concentrations. See Example 4.

FIG. 4 is a graph showing the results of the CHO-TIGIT CD155 blocking assay using human TIGIT-expressing CHO cells and the indicated TC-mAb™ mice-derived anti-TIGIT antibodies in the present invention at varying concentrations. See Example 5.

FIG. 5A is a graph showing the inhibitory effects of the indicated TC-mAb™ mice-derived anti-TIGIT antibody 4A063 in the present invention in combination with anti-PD1 antibody, on tumor growth in human TIGIT transgenic mouse model. FIG. 5B shows the average weight of mice each group as a function of time. See Example 6.

FIG. 6A is a graph showing the modulation of proportion of Tumor infiltrating lymphocyte CD8+ T (CD8+TIL) cells within CD3+ T cells population in CT26 tumor treated with the indicated TC mAb™-derived anti-TIGIT antibody 4A063 in combination with anti-PD1 antibody. See Example 7.

FIG. 6B is a graph showing the modulation of proportion of CD8+ T cells within CD3+ T cells population in spleen cells in the CT26 tumor bearing human TIGIT transgenic mice treated with the indicated TC mAb™-derived anti-TIGIT antibody 4A063 in combination with anti-PD1 antibody. See Example 7.

### DETAILED DESCRIPTION

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials are known in the art can also be used. The materials, methods, and examples are illustrative and not intended to be limiting. All publications, patent applications, patents, sequences, database entries, and other references mentioned herein are incorporated by reference in their entirety. In case of conflict, the present specification, including definitions, will control.

Although the numerical ranges and parameter approximations are shown in the broad scope of the present disclosure, the numerical values shown in the specific examples are described as accurately as possible. However, any numerical value inherently must contain a certain amount of error caused by the standard deviation present in their respective measurements. In addition, all ranges disclosed herein are understood to cover any and all subranges contained therein. For example, a range of “1 to 10” should be considered, including any and all subranges between a minimum of 1 and a maximum of 10 (inclusive); that is, all subranges beginning with a minimum of 1 or greater, such as 1 to 6.1, and subranges ending with a maximum of 10 or less, such as 5.5 to 10.

It should also be noted that, as used in this specification, the singular form includes the plural form of the object to which it refers unless it is clearly and explicitly limited to one object. The term “or” is used interchangeably with the term “and/or” unless the context indicates otherwise.

As used herein, the term “containing” or “comprising” means that various ingredients can be used together in the mixture or composition of the present invention. Therefore, the terms “mainly consisting of” or “consisting of” are included in the term “containing” or “comprising”.

The terms “identity”, “percent identity”, “homology” or “identical” as used herein refer to sequence identity between two amino acid sequences or between two nucleic acid sequences. Percent identity can be determined by aligning two sequences and refers to the number of identical residues (i.e., amino acids or nucleotides) at the positions shared by the compared sequences. Alignment and comparison of sequences can be performed using standard algorithms in the art (e.g., Smith and Waterman, 1981, Adv. Appl. Math. 2: 482; Needleman and Wunsch, 1970, J. Mol. Biol. 48: 443; Pearson and Lipman, 1988, Proc. Natl. Acad. Sci., USA, 85: 2444) or through computerized versions of these algorithms (Wisconsin Genetics Software Package Release 7.0, Genetics Computer Group, 575 Science Drive, Madison, WI), publically available as BLAST and FASTA. In addition, ENTREZ available at the National Institutes of Health (Bethesda MD) can be used for sequence comparison. When using BLAST or Gapped BLAST programs, the default parameters of each program (e.g., BLASTN, available on the Internet site of the National Center for Biotechnology Information) can be used. In one embodiment, GCG with a gap weight of 1 can be used to determine the percent identity between two sequences. Each amino acid gap is given weight as if it is a single amino acid mismatch between the two sequences. Alternatively, the ALIGN program (version 2.0) can be used, part of the GCG (Accelrys, San Diego, CA) sequence alignment software package.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

Traditional antibody structural units typically comprise a tetramer. Each tetramer is typically composed of two identical pairs of polypeptide chains, each pair having one “light” chain and one “heavy” chain. Human light chains are classified as kappa and lambda light chains. Heavy chains are classified as mu, delta, gamma, alpha, or epsilon, and define the antibody's isotype as IgM, IgD, IgG, and IgE, respectively. IgG has several subclasses, including, but not limited to IgG1, IgG2, IgG3, and IgG4. IgM has subclasses, including, but not limited to, IgM1 and IgM2. Thus, “isotype” as used herein is meant any of the subclasses of immunoglobulins defined by the chemical and antigenic characteristics of their constant regions. The known human immunoglobulin isotypes are IgG1, IgG2, IgG3, IgG4, IgA1, IgA2, IgM1, IgM2, IgD, and IgE. It should be understood that therapeutic antibodies can also comprise hybrids of isotypes and/or subclasses.

As used herein, “CDR region” or “CDR” refers to the hypervariable regions of the heavy and light chains of an immunoglobulin, as defined by Kabat et al. (Kabat et al., Sequences of proteins of immunological interest, 5th Ed., U.S. Department of Health and Human Services, NIH, 1991, and later). There are three heavy chain CDRs and three light chain CDRs. As used herein, the term CDR or CDRs is used to indicate one or several or even all of these regions, which contain most of the amino acid residues responsible for the binding through the affinity between an antibody and an antigen or epitope thereof.

As used herein, the term “antibody fragments” or “antigen-binding fragment” refers to a portion of a full-length antibody and antibody analogues of antibodies that retain the ability to specifically bind to an antigen (e.g., tigit), which usually include at least part of the antigen binding region or variable region of the parent antibody. In some embodiments, the antigen-binding fragment contains at least one variable domain (e.g., a variable domain of a heavy chain or a variable domain of light chain). The antibody fragment retains at least some binding specificity of the parent antibody. Generally, when activity is expressed in moles, antibody fragments retain at least 10% of maternal binding activity. Preferably, the antibody fragment retains at least 20%, 50%, 70%, 80%, 90%, 95% or 100% of the binding affinity of the parent antibody to the target. Antibody fragments include but are not limited to: Fab fragment, Fab′ fragment, F(ab′)2 fragment, Fv fragment, scFv fragment, FD fragment, complementary determination region (CDR) fragment, disulfide bond stability protein (dsFv), etc; Linear antibody, single chain antibody (e.g. scFv single antibody) (technology from Genmab), bivalent single chain antibody, single chain phage antibody, single domain antibody (e.g. VH domain antibody), domain antibody (technology from AbIynx); Multispecific antibodies formed from antibody fragments (e.g., three chain antibodies, four chain antibodies, etc.); And engineering modified antibodies, such as chimeric antibody (humanized mouse antibody), heteroconjugate antibody, etc. These antibody fragments are obtained by conventional techniques known to those skilled in the art, and the practicability of these fragments is screened by the same method as the complete antibody.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

In one embodiment, the antibodies of the invention can be multispecific antibodies, and notably bispecific antibodies, also sometimes referred to as “diabodies”. These are antibodies that bind to two (or more) different antigens, or different epitopes on the same antigen. Diabodies can be manufactured in a variety of ways known in the art, e.g., prepared chemically or from hybrid hybridomas.

As used herein, the term “Trans-chromosomic Mice (TC-mAb™ mice)” refers to the mice contains a mouse artificial chromosome which contains a human antibody heavy chain gene or locus, and/or a human antibody κ Light chain genes or loci, and/or human antibodies λ Light chain genes or loci, and at least two mouse endogenous antibody genes or loci corresponding to human antibody genes or loci are knocked out. The TC-mAb™ Mice and their offspring can stably maintain human antibody gene and produce human antibody.

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities.

The present invention further provides variant antibodies. That is, there are a number of modifications that can be made to the antibodies of the invention, including, but not limited to, amino acid modifications in the CDRs (affinity maturation), amino acid modifications in the Fc region, glycosylation variants, covalent modifications of other types, etc. For example, “variant” herein is meant a polypeptide sequence that differs from that of a parent polypeptide by virtue of at least one amino acid modification. Amino acid modifications can include substitutions, insertions and deletions. In general, variants can include any number of modifications, as long as the function of the protein is still present, as described herein.

As used herein, the terms “epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide. An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

As used herein, the terms “pharmaceutical composition,” “combined drug,” and “drug combination” are used interchangeably and mean a combination of at least one drug and optionally a pharmaceutically acceptable carrier or excipient used to achieve a specific purpose. In certain embodiments, the pharmaceutical composition includes combinations that are separated in time and/or space as long as they can work together to achieve the purpose of the present disclosure.

As used herein, a “therapeutically effective amount” or “effective amount” refers to a dose sufficient to exert its benefit to the subject being administered. The actual amount administered, as well as the rate and time course of administration, will depend on the condition of the person being treated and the severity. The prescription of treatment (e.g., dose determination, etc.) is ultimately the responsibility and decision of the GP and other doctors, usually considering the disease to be treated, the individual patient's condition, the delivery site, the method of administration, and other factors known to the doctor.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

The antibodies and chemotherapeutic agents of the invention are administered to a subject, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes.

Herein, the term “pharmaceutically acceptable” means that the compound is physiologically acceptable when the compound is administered to a human, and does not cause an allergic reaction such as a gastrointestinal disorder, dizziness or other allergic reaction, or a systemic allergic reaction similar to these allergic reactions.

In the present disclosure, “pharmaceutically acceptable carrier” includes, but is not limited to, binders (such as microcrystalline cellulose, alginates, gelatin and polyvinylpyrrolidone), fillers (such as starch, sucrose, glucose and anhydrous lactic acid), disintegrants (such as cross-linked PVP, cross-linked carboxymethyl sodium starch, croscarmellose sodium and low-substituted hydroxypropyl cellulose), lubricants (magnesium stearate, aluminum stearate, talc, polyethylene glycol, sodium benzoate), wetting agent (such as glycerin), surfactants (such as cetyl alcohol), and absorption enhancers, flavoring agents, sweeteners, diluents, coating agents, etc.

The term “TIGIT” or “T-cell immunoreceptor with Ig and ITIM domains” as used herein refers to any native TIGIT from any vertebrate source, including mammals such as primates (e.g., humans) and rodents (e.g., mice and rats), unless otherwise indicated TIGIT is also known in the art as DKFZp667A205, FLJ39873, V-set and immunoglobulin domain-containing protein 9, V-set and transmembrane domain-containing protein 3, VSIGU, VSTM3,and WUCAM. The term encompasses “full-length,” unprocessed TIGIT (e.g.,full-length human TIGIT having the amino acid sequence of SEQ ID NO: as well as any form of TIGIT. The term also encompasses naturally occurring variants of TIGIT, e.g., splice variants or allelic variants. The term “TIGIT-related diseases” refers to abnormal expression of TIGIT protein or its ligand CD155 in tumors (e.g., melanoma, breast cancer, non-small-cell lung carcinoma (NSCLC), colon adenocarcinoma (COAD), gastric cancer, acute myeloid leukaemia (AML) and multiple myeloma (MM) (Clin Exp Immunol. 2020 May;200 (2):108-119)), or immune-related disease (e.g., a T cell dysfunctional disorder diseases) in a subject (e.g., a human). After blocking its binding with ligands by anti-TIGIT antibody, the anti-TIGIT antibody can inhibit the growth of tumor cells, or alleviate symptoms of other disease, or cure related diseases, so as to achieve the therapeutic effect of diseases. Such diseases are defined as TIGIT-related diseases.

“T cell dysfunction disorder” is a T cell disorder or condition characterized by reduced responsiveness to antigenic stimuli. In some embodiments, the T cell dysfunctional disorder is characterized by T cell exhaustion. In a specific embodiment, a T cell dysfunction disorder is a disorder clearly associated with improperly reduced signal transduction via OX40 and/or OX40L. In another embodiment, a T cell dysfunction disorder is a disorder in which T cells are unresponsive or have a reduced ability to secrete cytokines, proliferate, or perform cell lytic activity. In a specific aspect, reduced responsiveness leads to ineffective control of immunogen expressing pathogens or tumors. Examples of T cell dysfunction disorders characterized by T cell dysfunction include unresolved acute infection, chronic infection, and tumor immunity. In some embodiments, the subject is a human.

The terms “cancer” and “tumor” are used interchangeably. They refer to a large class of diseases characterized by the uncontrolled growth of abnormal cells in the body. Uncontrolled cell division may lead to the formation of malignant tumors or cells invading adjacent tissues, and may be transferred to the distal part of the body through the lymphatic system or blood flow. Cancers include benign and malignant cancers as well as dormant tumors or micrometastasis. Cancer also includes hematological malignancies.

A “hematological malignancy” includes a lymphoma, leukemia, myeloma or a lymphoid malignancy, as well as a cancer of the spleen and the lymph nodes. Exemplary lymphomas include both B cell lymphomas and T cell lymphomas. B-cell lymphomas include both Hodgkin's lymphomas and most non-Hodgkin's lymphomas. Non-limiting examples of B cell lymphomas include diffuse follicular lymphoma, large B-cell lymphoma, mucosa-associated lymphatic tissue lymphoma, small cell lymphocytic lymphoma (overlaps with chronic lymphocytic leukemia), Burkitt's lymphoma, mantle cell lymphoma (MCL), mediastinal large B cell lymphoma, Waldenstrom macroglobulinemia, nodal marginal zone B cell lymphoma, splenic marginal zone lymphoma, primary effusion lymphoma, intravascular large B-cell lymphoma, lymphomatoid granulomatosis. Non-limiting examples of T cell lymphomas include extranodal T cell lymphoma, cutaneous T cell lymphomas anaplastic large cell lymphoma, and angioimmunoblastic T cell lymphoma. Hematological malignancies also include leukemia, such as, but not limited to, secondary leukemia, chronic lymphocytic leukemia, acute myelogenous leukemia, chronic myelogenous leukemia, and acute lymphoblastic leukemia. Hematological malignancies further include myelomas, such as, but not limited to, multiple myeloma and smoldering multiple myeloma. Other hematological and/or B cell- or T-cell-associated cancers are encompassed by the term hematological malignancy.

The term “PVR positive tumors” refers to tumors that the expression of PVR is increased in cancer tissues. PVR positive tumors include but are not limited to Provide adrenocortical carcinoma, chromophobe renal cell carcinoma, papillary kidney carcinoma, liver hepatocellular carcinoma, pancreatic ductal adenocarcinoma, pheochromocytoma & paraganglioma, lung adenocarcinoma, head and neck squamous cell carcinoma prostate adenocarcinoma, uterine corpus endometrial carcinoma, cervical carcinoma, and cutaneous melanoma, mesothelioma, urethral epithelial bladder cancer, colon and rectal adenocarcinoma, clear cell kidney carcinoma, lung squamous cell carcinoma, uterine carcinosarcoma, sarcoma, ovarian serous cystadenocarcinoma, papillary thyroid carcinoma, glioblastoma multiforme, breast cancer, lower grade glioma and diffuse large B-cell lymphoma.

Herein, the term “immune related diseases” refers to immune related diseases in mammals caused, mediated, or otherwise contributed to by components of the mammalian immune system, and also includes diseases that stimulate or interfere with the immune response can improve the development of the disease. The “immune related diseases” includes immunological mediated inflammatory diseases, non-immune mediated inflammatory diseases, infectious diseases, immunodeficiency diseases, tumors, etc.

The anti TIGIT antibody or its antigen binding fragment of the invention can be used to treat infections or infectious diseases in subjects (such as human beings). In some preferred implementation options, the infectious or infectious diseases are selected from viral infections, bacterial infections, fungal infections and parasitic infections, including but not limited to HIV, hepatitis virus, herpes virus, CMV, EBV, influenza.

In the following, some preferred embodiments and aspects of the present invention will be further described in conjunction with specific examples, and these examples should not be construed as limiting the scope of the present invention.

### EXAMPLES

### Example 1 Generation of Anti-TIGIT Monoclonal Antibodies

### 1. TIGIT Recombinant Protein for Antigen Immunization and Binding Assay

The cDNA encoding full-length human TIGIT (huTIGIT, SEQ ID NO: 1) based on its GenBank sequence (Locus: NM_173799) was synthesized and purchased from Eurofins. The coding region of the extracellular domain ECD corresponding to amino acid (AA) 1-141 (SEQ ID NO: 2) of full-length human TIGIT was amplified by PCR and cloned into the expression vector to produce two recombinant fusion proteins Trx-huTIGIT-HIS and Gst-huTIGIT-HIS expression plasmids, respectively. For the production of recombinant fusion protein, the recombinant fusion protein (Trx-TIGIT-HIS and Gst-TIGIT-HIS) expression plasmids were transferred into competent E.coli (E.coli gamiB (DE3)pLysS, Novagen) for culture. After IPTG induction, E.coli cells were centrifuged to collect precipitate. After ultrasonic crushing of E.coli, the precipitate was obtained by centrifugation. Solubilizing reagent was added to dissolve the precipitate, and then purified by Ni-NTA column (Qiagen, Ni-NTA Superflow, #30410) and dialyzed. The purification effect of recombinant protein was detected by PAGE, and then the protein was stored at −30° C. as small aliquots.

(SEQ ID NO: 1)
ATGCGCTGGTGTCTCCTCCTGATCTGGGCCCAGGGGCTGAGGCAGGCTC
CCCTCGCCTCAGGAATGATGACAGGCACAATAGAAACAACGGGGAACAT
TTCTGCAGAGAAAGGTGGCTCTATCATCTTACAATGTCACCTCTCCTCC
ACCACGGCACAAGTGACCCAGGTCAACTGGGAGCAGCAGGACCAGCTTC
TGGCCATTTGTAATGCTGACTTGGGGTGGCACATCTCCCCATCCTTCAA
GGATCGAGTGGCCCCAGGTCCCGGCCTGGGCCTCACCCTCCAGTCGCTG
ACCGTGAACGATACAGGGGAGTACTTCTGCATCTATCACACCTACCCTG
ATGGGACGTACACTGGGAGAATCTTCCTGGAGGTCCTAGAAAGCTCAGT
GGCTGAGCACGGTGCCAGGTTCCAGATTCCATTGCTTGGAGCCATGGCC
GCGACGCTGGTGGTCATCTGCACAGCAGTCATCGTGGTGGTCGCGTTGA
CTAGAAAGAAGAAAGCCCTCAGAATCCATTCTGTGGAAGGTGACCTCAG
GAGAAAATCAGCTGGACAGGAGGAATGGAGCCCCAGTGCTCCCTCACCC
CCAGGAAGCTGTGTCCAGGCAGAAGCTGCACCTGCTGGGCTCTGTGGAG
AGCAGCGGGGAGAGGACTGTGCCGAGCTGCATGACTACTTCAATGTCCT
GAGTTACAGAAGCCTGGGTAACTGCAGCTTCTTCACAGAGACTGGTTA
G;
(SEQ ID NO: 2)
MMTGTIETTGNISAEKGGSIILQCHLSSTTAQVTQVNWEQQDQLLAICN
ADLGWHISPSFKDRVAPGPGLGLTLQSLTVNDTGEYFCIYHTYPDGTYT
GRIFLEVLESSVAEHGARFQIP.

### 2. Establishment of Stable Expression Cell Lines

The cDNA encoding full-length human TIGIT (huTIGIT, SEQ ID NO: 1) and cynomolgus monkey TIGIT (mkTIGIT, SEQ ID NO: 3) based on their GenBank sequences (NM_73799) and (XM_005548101.2) were synthesized and purchased from the Genscript, respectively. After PCR amplification, the DNA products were cloned into pcDNA3.1 expression vector (Invitrogen) and transduced into CHO-K1 cell line (JCRB, #JCRB9018) to produce CHO-huTIGIT and CHO-mkTIGIT cell lines. HuTIGIT or mkTIGIT high expression stable cell lines were selected by culture in medium with G418, eGFP expression and FACS binding assay.

(SEQ ID NO: 3)
ATGCGGTGGTGTCTCTTCCTGATCTGGGCCCAGGGGCTGAGGCAGGCTC
CCCTCGCCTCAGGAATGATGACAGGCACAATAGAAACAACGGGGAACAT
TTCTGCAAAGAAAGGTGGCTCTGTTATCTTACAATGTCACCTCTCCTCC
ACCATGGCACAAGTGACCCAGGTCAACTGGGAGCAGCATGACCATTCGC
TTCTGGCCATTCGTAATGCTGAGTTGGGGTGGCACATCTACCCAGCCTT
CAAGGATCGAGTGGCCCCGGGTCCTGGCCTGGGCCTCACCCTCCAGTCG
CTGACCATGAATGATACAGGGGAGTACTTCTGCACCTATCACACCTACC
CTGATGGGACTTACAGAGGGAGAATCTTCCTGGAGGTCCTAGAAAGCTC
AGTGGCTGAGCACAGTGCCAGGTTCCAGATTCCATTGCTTGGAGCCATG
GCCATGATGCTGGTGGTCATCTGCATAGCAGTCATCGTGGTGGTCGTGT
TGGCTAGAAAGAAGAAATCCCTCAGAATCCATTCTGTGGAAAGTGGCCT
CCAGAGAAAATCAACTGGACAGGAAGAACAGATTCCCAGTGCTCCCTCA
CCCCCAGGAAGCTGTGTCCAGGCAGAAGCTGCACCTGCTGGGCTCTGTG
GAGAGCAGCAGGGAGATGACTGTGCCGAGCTGCATGACTACTTCAATGT
CCTGAGTTACAGAAGCCTGGGGAGCTGCAGCTTCTTCACAGAGACTGGG
TAG.

### 3. Immunization, Hybridoma Fusion and Cloning

Human TIGIT recombinant protein (Trx-huTIGIT-HIS fusion protein, 100 μg/mouse/primary, 50 μg/mouse/boost) was mixed with Freund's complete adjuvant (FCA, purchased from BD, Cat. 263810, 100 μL/mouse/primary) for the primary immune injection of 6-8-week-old trans-chromosomic mice (TC-mAb™ Mice), and (Sigma Adjust System®,SAS, purchased from sigma, Cat.s6322-1vl, 50 μL/mouse/boost) for the boost immune injections was used, with an interval of 2-3 weeks. Adjuvant is not required for the last immunization, and only Trx-huTIGIT-HIS fusion protein (50 μg/mouse/final) was needed. All immunization was performed by intraperitoneal injection.

Three days after the last immunization, the mice were euthanized, the spleen and lymph nodes were aseptically removed, and the mouse lymphocytes were aseptically isolated and extracted. The obtained lymphocyte population was fused with mouse myeloma cells (1:1, P3X63-Ag8.653, ATCC, #CRL-1580) by electrofusion. The fused cells were placed in 96 well plates in the HAT medium, incubated at 37° C. and 5% CO₂ for 7 days, changed to the HT medium, and incubated for 5 days.

### 4. Evaluation of Binding Activity of Anti-TIGIT Antibodies by ELISA, Immunocytochemistry (ICC) and Flow Cytometry

The supernatant contained specific anti-TIGIT antibodies was screened using human TIGIT protein by ELISA. 96-well plates (Nunc, cat. 44-2404) were coated with Gst-huTIGIT-HIS fusion protein and Trx-huTIGIT-HIS (50 ng/well), respectively, diluted with PBS buffer and incubated overnight at 4° C. Then 300 μL TBS containing skim milk and Tween-20 was used to seal the wells at room temperature for 0.5 hours. After washing, added 100 μL supernatant and/or serum and incubated at room temperature. To detect the specificity of antibodies, horseradish peroxidase conjugated anti-human IgG antibodies (Goat anti-Human IgG-Fc Fragment cross-adsorbed Antibody HRP Conjugated, BETHYL, #A80-304P) were diluted to an optimized concentration in PBS containing 0.05% Tween-20 and then was added as 100 μL/well after washing, incubated at room temperature for 0.5 hours. The plate was washed using 300 μL TBS containing 0.05% Tween-20 for 3 times. Add 100 μL matrix solution containing 0.5 mg/mL OPD and 0.03% H₂O₂, incubate the plate at room temperature for 30 minutes, and then add 25 μL 1M H₂SO₄ (Nacalai Tesque, #95626-06), and then read at 492 nm. The positive wells clone was selected and seeded into the new 96 well plate. After 3 days, the supernatant of the new 96 well plate was screened using human TIGIT protein by ELISA. Human TIGIT-binding hybridoma cell lines were expanded and cultured for 2-4 days, CHO-huTIGIT and CHO-mkTIGIT cells were used to detect the ELISA positive clones by ICC and flow cytometry, and the positive clones were selected. After several days of culture, the secondary detection was carried out according to the above method. The secondary positive clones were diluted to the limit, ICC and flow cytometry were tested again two weeks later, and the limit dilution was carried out again.

### Example 2 Sequence Analysis of Anti-TIGIT Antibodies

After preliminary screening by ELISA, ICC and FACS, the positive hybridoma clones were subcloned by limited dilution. After validation again, the cloned hybridoma cells were cultured in a 10 cm dish. When the cell density reached 80% to 90%, we collected the cells and suspended them with solution. RNA was extracted from suspension cells with a microKit (QIAGEN, #74104). The extracted RNA was rapidly amplified at the 5′cDNA end using a kit (TaKaRa, #Z4858N). The sequence analysis of products (Eurofins) is shown in Table 1.

According to the sequence, the TIGIT antibody expression plasmid was constructed and was expressed in HEK293 cells. Eight antibodies were purified by protein A, and the antibodies purity was more than 95% by SDS-PAGE analysis. The ten antibodies obtained were subjected to amino acid sequencing, and the heavy chain variable region (VH) and light chain variable region (VL) sequences are shown in Table 1.

TABLE 1
Variable region sequences of VH and VL regions of the TC-mAb ™
Mice-derived anti-TIGIT antibodies in the present invention.

| | | | SEQ ID |
|---|---|---|---|
| Ab | VL/VH | Amino Acid Sequence | NO: |
| JHH1B2-8C | VL | DIVMTQSPDSLAVSLGERATINCKSSQSVLYSSNNKNYLAWYQQKPGQ | 4 |
| | | PPKLLIYWASTRESGVPDRFSGSGSGTDFTLTISSLQAEDVAVYYCQQYY | |
| | | STPWTFGQGTKVEIK | |
| | VH | QVQLQQSGPGLVKPSQTLSLTCAISGDSVSSNSAAWNWIRQSPSRGLE | 5 |
| | | WLGRTYYRSKWYNDYAVSVKSRITINPDTSKNQFSLQLNSVTPEDTAVY | |
| | | YCAREGVLRYFDWLSGAFDIWGQGTMVTVSS | |
| JHH4A042-H3 | VL | DIVMTQSPDSLAVSLGERATINCKSSQSVLFGFNNKIYLAWYQQKPGQ | 6 |
| | | PPELLIYWASTRESGVPDRFSGSGSGTDFTLTISSLQAEDVAVYYCQQYY | |
| | | SFPYTFGQGTKLEIK | |
| | VH | QVQLQQSGPGLVKPSQTLSLTCAISGDSVSSNSAAWNWIRQSPSRGLE | 7 |
| | | WLGRTYFRSKWYNDYAVSVKSRITINPDTSKNQFSLQLNSMTPEDTAV | |
| | | YYCARERALYDILAGSFDYWGQGTLVTVSS | |
| JHH4A042-H7 | VL | DIVMTQSPDSLAVSLGERATINCKSSQSVLFGFNNKIYLAWYQQKPGQ | 8 |
| | | PPELLIYWASTRESGVPDRFSGSGSGTDFTLTISSLQAEDVAVYYCQQYY | |
| | | SFPYTFGQGTKLEIK | |
| | VH | QVQLQQSGPGLVKPSQTLSLTCAISGDSVSSNSAVWNWIRQSPSRGLE | 9 |
| | | WLGRTYYRSKWYNDYAVSVKSRITINPDTSKNQFSLQLNSMTPEDTAV | |
| | | YYCARERALYDILAGSFDYWGQGTLVTVSS | |
| JHH4B030a | VL | DIVMTQSPDSLAVSLGERATINCKSSQSVLYSSNNKNYLAWYQQKPGQ | 10 |
| | | PPKLLIYWASTRESGVPDRFSGSGSGTDFTLTISSLQAEDVAVYYCQQHY | |
| | | SFPYTFGQGTTLEIK | |
| | VH | QVQLQQSGPGLVKPSQTLSLTCAISGDSVSSNSAAWNWIRQSPSRGLE | 11 |
| | | WLGRTYYRSKWYNDYAVSVKSRITINPDTSKNQFSLQLNSVTPEDTAVY | |
| | | YCARERGTYYDILTGDFDYWGQGTLVTVSS | |
| JHH4B037a | VL | DIVMTQSPLSLPVTPGEPASISCRSSQSLLHSNGYNYLDWFLQKPGQSP | 12 |
| | | QLLIYLGSNRASGVPDRFSGSGSGTDFTLKISRVGAEDVGVYYCMQAL | |
| | | QTPLTFGGGTKVEIK | |
| | VH | EVQLVESGGGLVQPGRSLRLSCEASGFSFDDYAMHWVRQAPGKGLE | 13 |
| | | WVSGISWNSVTIGYADSVKGRFTISRDNAKNSLYLEMNSLRTEDTALYY | |
| | | CAKELPTYRYYALDGWGQGTAVTVSS | |
| JHH4B056a | VL | DIVMTQSPLSLPVTPGEPASISCRSSQSLLHSNGYKYLNWYLQKPGQSP | 14 |
| | | QLLIYLGSNRASGVPDRFSGSGSGTDFTLKISRVEAEDVGVYYCMQALQ | |
| | | TPLTFGGGTKVEIK | |
| | VH | EVQLVESGGGLVQPGRSLRLSCAASGFTFDDYAMHWVRQAPGKGLE | 15 |
| | | WVSGISWNGIGIGYADSVKGRFTISRDNAKNSLYLQMNSLRAEDTALYY | |
| | | CAKDGGDYHYYGMDVWGQGTTVTVSS | |
| JHH4A057 | VL | EIVLTQSPGTLFLSPGERATLSCRASQSVSRNYLVWYQQKPGQAPRLLIY | 16 |
| | | GASTRATGIPDRFSGSGSGTDFTLTIRRLEPEDFALYYCQQYGSSPFTFG | |
| | | QGTKLEIN | |
| | VH | QVQLVQSGSELKKPGASVKVSCKASGYTLTSHALNWVRQAPGQGLE | 17 |
| | | WMGWINTNTGNPTYVQGFTGRFVFSLDTSVSTAYLQISSLKAEDTAVY | |
| | | YCARGYSSPPRMDVWGQGTTVTVSS | |
| JHH4A063 | VL | DIMMTQSPLSLPVTPGEPASISCRSSQSLLHSNGYKYLNWYLQKPGQS | 18 |
| | | PLLLIYLGSNRASGVPDRFSGSGSGTDFTLKISRVEAEDVGVYYCMQAL | |
| | | QTPLTFGGGTKVEIK | |
| | VH | EVQLVESGGGLVQPGRSLRLSCTASGFNFDDYGMHWVRQAPGKGLE | 19 |
| | | WVSGISWNSISIGSADSVKGRFTISRDNAKNSLYLQMNSLRPEDTALYY | |
| | | CAKDGGDHYYYGMDVWGQGTTVTVSS | |
| JHH4D035a | VL | DIVMTQSPLSLPVTPGEPASISCRSSQSLLHSNGYNYLDWYLQKPGQSP | 68 |
| | | QLLIYLGSNRASGVPDRFSGSGSGTDFTLKISRMEAEDVGVYYCMQAL | |
| | | QTPPTFGGGTKVEIR | |
| | VH | EVQLVESGGGLVQPGRSLRLSCAASGFTFDDFAMHWVRQAPGKGLE | 69 |
| | | WVSGISWNGIGIGYADSVKGRFTISRDNAKNSLYLQMNSLRAEDTALYF | |
| | | CAKDKGLRRYYGMDVWGQGITVIVSS | |
| JHH4E061a | VL | EIVMTQSPLSLPVTPGEPASISCRSSRSLLHSNGYNYLDWYLQKPGQSP | 70 |
| | | QLLIYLGSNRASGVPDRFSGSGSGTDFTLKINRVEAEDVGVYYCMQAL | |
| | | QTPPTFGGGTKVEIK | |
| | VH | EVQLVESGGGLIQPGRSLTLSCVASGFTFDDYAMHWVRQAPGKGLEW | 71 |
| | | VSGISWNGIGIGYADSVKGRFTISRDNAKNSLILQMNSLRIEDTALYYCA | |
| | | KDKGLRRYYGMDVWGQGTTVTVSS | |

The IMGT program was used to perform CDR prediction on VL and VH, and the results are shown in Table 2.

TABLE 2
Predicted CDRs of the eight antibodies

| | | | SEQ | | SEQ | | SEQ |
|---|---|---|---|---|---|---|---|
| | | | ID | | ID | | ID |
| Ab | | CDR1 | NO: | CDR2 | NO: | CDR3 | NO: |
| JHH1B2-8C | VL | QSVLYSSNNKNY | 20 | WAS | 21 | QQYYSTPWT | 22 |
| | VH | GDSVSSNSAA | 23 | TYYRSKWYN | 24 | AREGVLRYFDWLSGAFDI | 25 |
| JHH4A042-H3 | VL | QSVLFGFNNKIY | 26 | WAS | 27 | QQYYSFPYT | 28 |
| | VH | GDSVSSNSAA | 29 | TYFRSKWYN | 30 | ARERALYDILAGSFDY | 31 |
| JHH4A042-H7 | VL | QSVLFGFNNKIY | 32 | WAS | 33 | QQYYSFPYT | 34 |
| | VH | GDSVSSNSAV | 35 | TYYRSKWYN | 36 | ARERALYDILAGSFDY | 37 |
| JHH4B030a | VL | QSVLYSSNNKNY | 38 | WAS | 39 | QQHYSFPYT | 40 |
| | VH | GDSVSSNSAA | 41 | TYYRSKWYN | 42 | ARERGTYYDILTGDFDY | 43 |
| JHH4B037a | VL | QSLLHSNGYNY | 44 | LGS | 45 | MQALQTPLT | 46 |
| | VH | GFSFDDYA | 47 | ISWNSVTI | 48 | AKELPTYRYYALDG | 49 |
| JHH4B056a | VL | QSLLHSNGYKY | 50 | LGS | 51 | MQALQTPLT | 52 |
| | VH | GFTFDDYA | 53 | ISWNGIGI | 54 | AKDGGDYHYYGMDV | 55 |
| JHH4A057 | VL | QSVSRNY | 56 | GAS | 57 | QQYGSSPFT | 58 |
| | VH | GYTLTSHA | 59 | INTNTGNP | 60 | ARGYSSPPRMDV | 61 |
| JHH4A063 | VL | QSLLHSNGYKY | 62 | LGS | 63 | MQALQTPLT | 64 |
| | VH | GFNFDDYG | 65 | ISWNSISI | 66 | AKDGGDHYYYGMDV | 67 |
| JHH4D035a | VL | QSLLHSNGYNY | 72 | LGS | 73 | MQALQTPPT | 74 |
| | VH | GFTFDDFA | 75 | ISWNGIGI | 76 | AKDKGLRRYYGMDV | 77 |
| JHH4E061a | VL | RSLLHSNGYNY | 78 | LGS | 79 | MQALQTPPT | 80 |
| | VH | GFTFDDYA | 81 | ISWNGIGI | 82 | AKDKGLRRYYGMDV | 83 |

It should be noted that when using different CDR prediction programs, the CDRs of the same VH or the same VL may show slight differences, such as changes in amino acid positions. These different CDRs based on the same VH or VL are also within the scope of the present invention.

### Example 3 Determination of Affinity Between the Indicated TC-mAb™ Mice-Derived Anti-TIGIT Antibodies and Recombinant Human TIGIT Protein

The equilibrium dissociation constant (KD) of the eight example antibodies mentioned above of the invention binding to human TIGIT is measured by FortebioOctet RED96. The measurement method is carried out according to the existing method (Estep, P et al., High throughput solution Based measurement of antibody-antigen affinity and epitope binning, MAbs, 2013. 5(2):p.270-8). The affinity between the indicated TC-mAb™ Mice-derived anti-TIGIT antibody in the present invention and TIGIT-HIS (Biointron, BI120) was measured. NTA (HIS-tag) sensor was applied. After the sensor was balanced in the analysis buffer, human TIGIT-HIS was loaded onto NTA sensor (fortebio) for affinity measurement. The sensor loaded with antigen was placed in the solution containing antibody (the antibody concentrations were 5, 2.5, 0.83, 0.278, 0.09, 0.03 and 0.01 μg/ml respectively) until the plateau period, and then transfer the sensor to the analytical buffer for dissociation for at least 2 minutes for dissociation rate measurement. The 1:1 combined model was used for dynamic analysis.

In the experiment as described above, the KD values of the indicated TC-mAb™ Mice-derived anti-TIGIT antibodies in the present invention are shown in Table 3.

TABLE 3
Monovalent KD of eight antibodies

| | | Dissociation Time | | | | | | | |
|---|---|---|---|---|---|---|---|---|---|
| Ab | Binding Time | 60 s | 120 s | 150 s | 200 s | 300 s | 600 s | 900 s | 1200 s |
| JHH1B2-8C | 240 s | 2.42E−06 | 2.434E−07 | 1.013E−07 | 1.559E−08 | 6.449E−09 | 9.546E−10 | 5.079E−10 | — |
| JHH4A042-H3 | 300 s | 4.55E−09 | 1.31E−09 | 9.09E−10 | 5.40E−10 | 2.59E−10 | 8.99E−11 | 5.02E−11 | — |
| JHH4A042-H7 | 240 s | 1.995E−10 | 1.314E−10 | 1.198E−10 | 1.08E−10 | 7.701E−11 | 5.177E−11 | — | — |
| JHH4B030a | 240 s | 7.15E−09 | 2.50E−09 | 1.90E−09 | 1.33E−09 | 7.10E−10 | 1.79E−10 | 7.05E−11 | — |
| JHH4B037a | 240 s | 3.51E−10 | — | — | 5.21E−11 | — | 1.4E−11 | 1.976E−11 | — |
| JHH4B056a | 240 s | — | — | — | — | 3.08E−10 | 5.77E−11 | 2.00E−10 | — |
| JHH4A057 | 240 s | 1.699E−09 | 9.363E−10 | 8.216E−10 | 6.00E−10 | 4.19E−10 | 2.724E−10 | 1.962E−10 | 1.51E−10 |
| JHH4A063 | 300 s | 5.51E−09 | 1.54E−09 | 1.06E−09 | 5.32E−10 | 1.99E−11 | — | — | — |

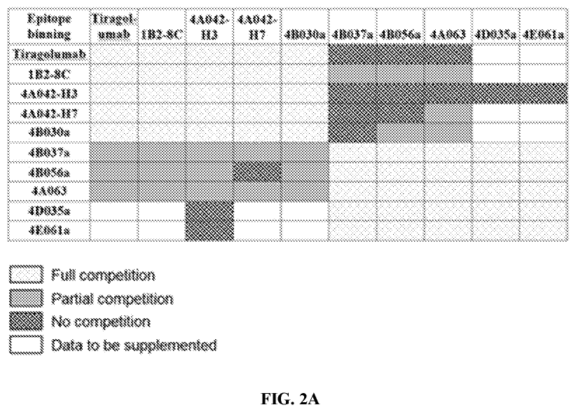

Compared with the anti-TIGIT reference antibody Tiragolumab (synthesized according to the Tiragolumab sequence disclosed in KEGG-DRUG database), the epitopes of all human TIGIT antibodies (1B2-8C, 4A042-H3, 4A042-H7, 4B030a, 4B037a, 4B056a, 4A063, 4D035a, 4E061a) bound to human TIGIT were studied using Octet binding test. The experimental process was as follows: the sensor loaded with TIGIT-HIS (Biointron, BI120) is placed in the solution containing TIGIT antibodies until the platform stage, and then the sensor is transferred to an analytical buffer to saturation, and then the sensor is transferred to other analytes or buffer containing the reference antibody (Tiragolumab). After binding to the platform stage, it is eluted. The epitope grouping showed that there was competition among the three candidate clonal epitopes, that is, they combined with the same antigen epitope of TIGIT, compared with Tiragolumab. The results are shown in FIG. 2A.

There was epitope competition among various antibodies in the present invention. 1B2-8C can block the binding of itself and Tiragolumab, 4A042-H3, 4A042-H7 and 4B030a, partially block the binding of 4A063, but not 4B037a, 4B056a, 4A063, 4D035a and 4E061a; Tiragolumab and 4A042-H3 can block the binding of Tiragolumab, 1B2-8C, 4A042-H7 and 4B030a without blocking the binding of 4B037a, 4B056a, 4A063, 4D035a and 4E061a; 4A042-H7 can block the binding of itself and Tiragolumab, 1B2-8C, 4A042-H3 and 4B030a, partially block the binding of 4B037a and 4A063, but not 4B056a, 4D035a and 4E061a; 4B030a can block the binding of itself, partially block the binding of 4B056a and 4A063, and do not block the binding of 4B037a, 4D035a and 4E061a; 4B037a and 4A063 can block the binding of 4B037a, 4B056a, 4A063, 4D035a and 4E061a, and partially block the binding of Tiragolumab, 1B2-8C, 4A042-H3, 4A042-H7 and 4B030a; 4B056a can block the binding of itself and 4B037a, 4A063, 4D035a and 4E061a, partially block the binding of Tiragolumab, 1B2-8C, 4A042-H3 and 4B030a, but not 4A042-H7; 4D035a and 4E061a can block the binding of 4B037a, 4B056a, 4A063, 4D035a and 4E061a without blocking the binding of Tiragolumab, 1B2-8C, 4A042-H3, 4A042-H7 and 4B030a. The results are shown in FIG. 2B and 2C.

### Example 4 Binding Activity of the Indicated TC-mAb™ Mice-Derived Anti-TIGIT Monoclonal Antibodies to Cell Surface TIGIT

CHO-huTIGIT cells or CHO-mkTIGIT cells were implanted in 96 well plates. The antibodies of Example 2 were diluted to different concentrations respectively and added to 96 well plates covered with cells (100 μL/well), incubated at 4° C. (on ice) for 1 hour.

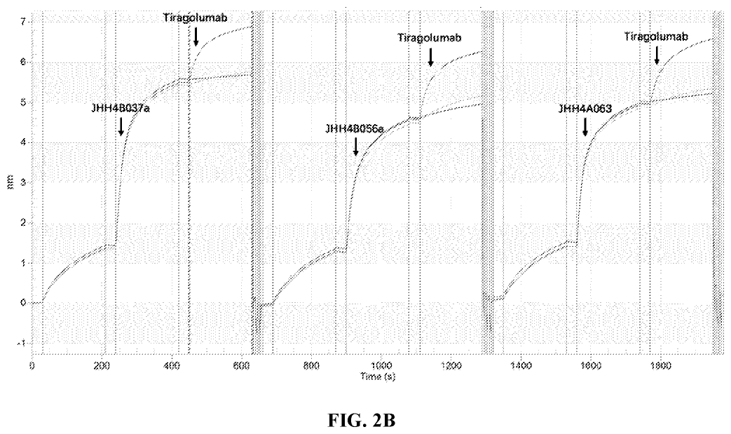

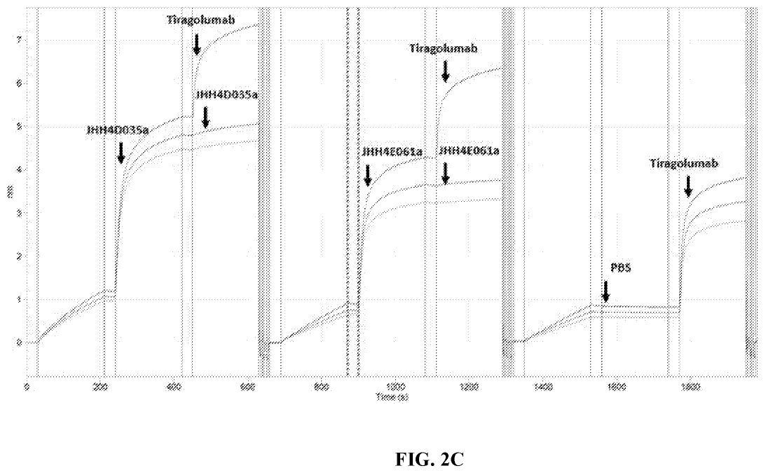

Washed the cells by adding 200 μL/well washing buffer, and centrifuged at 1600 rpm (˜260×g), 4° C. for 3 minutes, and discarded the supernatant, repeated twice. Add 30 μL washing buffer containing anti-human IgG secondary antibody (Jackson ImmunoResearch, #109-585-190, Alexa Fluor® 594 AffiniPure Goat Anti-Human IgG, Fcγ fragment specific) to each well, cultured cells at 4° C. (on ice) for 1 hour. Washing twice, transferred the cells to a flat bottom 96 well plate, and analyze the cells with CytoFLEX S. The antibody concentration was plotted with the logarithm of 10 as the abscissa and the median fluorescence value of two channels as the ordinate. The EC₅₀ (CHO-huTIGIT and CHO-mkTIGIT) and the peak value of the curve were compared. The (μg/mL) results are shown in Table 4 and FIG. 3A-3B.

TABLE 4
Binding activity (EC₅₀, μg/mL) between the antibodies
and human TIGIT or cynomolgus monkey TIGIT

| | EC₅₀ | EC₅₀ |
|---|---|---|
| Ab | (CHO-huTIGIT) | (CHO-mkTIGIT) |
| IgG1 | 385358 | — |
| JHH1B2-8C | 0.005489 | 573.5872 |
| JHH4A042-H3 | 0.004629 | 0.079222 |
| JHH4A042-H7 | 0.006025 | 0.604994 |
| JHH4B030a | 0.004298 | 0.857118 |
| JHH4B037a | 0.003882 | 0.005315 |
| JHH4B056a | 0.004594 | 0.009579 |
| JHH4A057 | 0.004118 | 0.008868 |
| JHH4A063 | 0.003494 | 0.004251 |

### Example 5 TC-mAb™ Mice-Derived Anti-TIGIT Monoclonal Antibodies in the Present Invention Block the Binding of TIGIT and CD155

CHO-huTIGIT-eGFP cells were implanted in V-bottom 96 well plates. The antibodies of Example 2 and reference antibodies were diluted to different concentrations respectively and added (100 μL/well) to the 96 well plate covered with cells (100%)), incubated at 4° C. for 1 hour.

Washed the cells by adding 200 μL/well washing buffer, and centrifuged at 1600 rpm (˜260×g), 4° C. for 3 minutes, and discarded the supernatant, repeated twice. Add 30 μL washing buffer containing biotinylated human CD155 (Human CD155/PVR/NECL5 Protein (Fc Tag), Biotinylated, Sino Biological, 10109-H02H-B), then the cells were cultured at 4° C. (on ice) for 1 hour. Add 30 μL washing buffer contains streptavidin-594 (f.c.=10 μg/ml) into the well, then the cells were incubated at 4° C. for 30 minutes. Wash once and transfer the cells to a flat bottom 96 well plate, and analyze the cells with CytoFLEX S. Take the logarithm of antibody concentration (μg/mL) with the base of 10 as the abscissa and the intermediate fluorescence value corresponding to each antibody concentration as the ordinate. The blocking ability of different antibodies to block the binding between CD155 and TIGIT was distinguished by analyzing the IC₅₀ of the curve.

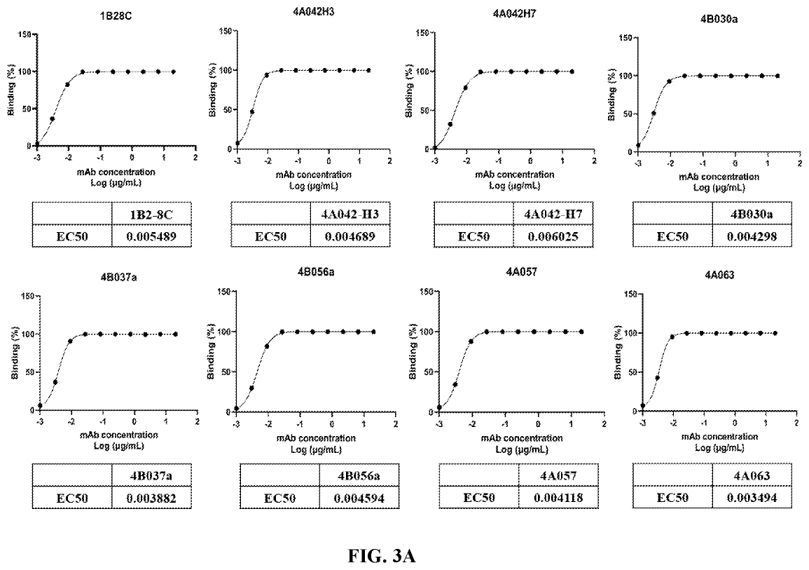

Taking IgG1 as negative control antibody, the blocking ability of the eight antibodies to CD155 was tested. The IC₅₀ (μg/mL) results are shown in Table 5 and FIG. 4.

TABLE 5
Blocking ability (IC₅₀) of the indicated TC-mAb ™ Mice-derived
anti-TIGIT monoclonal antibodies to block the binding
of CD155 with TIGIT

| Ab | IC₅₀ |
|---|---|
| IgG1 | — |
| JHH1B2-8C | 1.181205 |
| JHH4A042-H3 | 0.800423 |
| JHH4A042-H7 | 0.972927 |
| JHH4B030a | 0.776606 |
| JHH4B037a | 0.7898 |
| JHH4B056a | 1.002 |
| JHH4A057 | 0.8995 |
| JHH4A063 | 0.5588 |

Example 6 In vivo efficacy of combination therapy of anti-TIGIT and anti-PD-1 antibody

To evaluate the synergistic effect of anti-TIGIT and anti-PD-1 antibodies in vivo, mouse colorectal cancer cell line CT26. WT (5×10⁵ cells/mouse) were transplanted subcutaneously into TIGIT humanized BALB/c mice. When the average tumor volume reached 120±50 mm³, the mice were randomly divided according to the tumor volume (n=3), and IgG negative control antibody (Anti-HEL-Human IgG1 Isotype, biointron, 200 μg/mouse/time), anti-mPD-1 antibody (InVivoMAb anti-mouse PD-1, lot: 795720D1, 20 μg/mouse/time), anti-mPD-1 antibody (20 μg/mouse/time) combined with positive reference antibody (Tiragolumab-hIgG1, 200 μg/mouse/time), anti-mPD-1 antibody (20 μg/mouse/time) combined with 4A063 antibody (4A063-hIgG1, 200 μg/mouse/time) were administered every three days, and totally for 6 times. Tumor volume and body weight was measured twice a week.

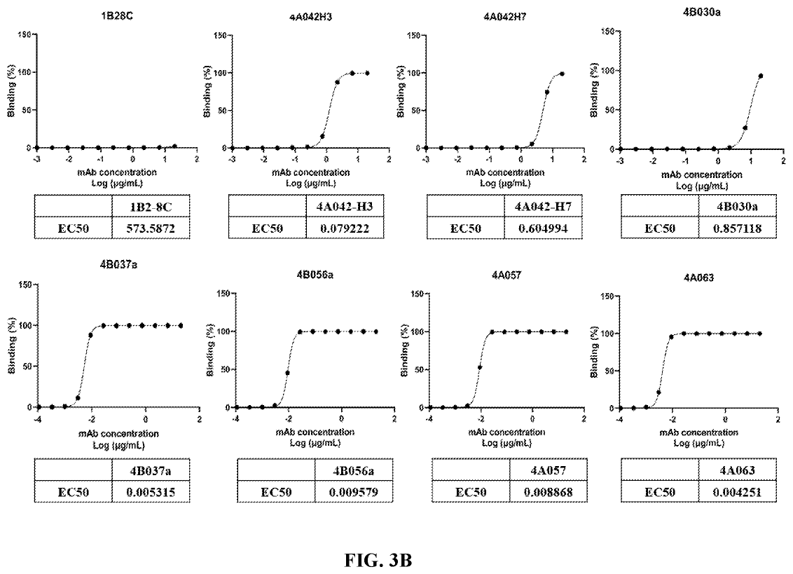

The results are shown in FIG. 5, which provides the average tumor volume of each group as a function of time and the average weight of each group as a function of time. Compared with the positive reference antibody, 4A063 showed significant inhibition on tumor growth (TGI: 44% vs 92.33%), and there was no significant difference in average body weight among the groups.

### Example 7 Characterization of the Mechanism of Action of In Vivo Anti-Tumor Activity of Combination Therapy of Anti-TIGIT and Anti-PD-1 Antibody

To investigate the in vivo mode of action of anti-TIGIT antibody, tumors were analyzed by flow cytometry for the immune cell infiltrate following combination treatment with anti-TIGIT antibody 4A063 (hIgG1) and anti-PD-1 antibody. Mice were inoculated and treated as described in example 6. Three days after the six treatment, mice were sacrificed and tumors and spleen harvested. Tumors were dissociated with a tumor digestion buffer (1 mg/mL CollagenaseI and 20 μg/mL DNAase I, sigma) and spleen single cell suspension was obtained directly after spleen grinding. Cell were stained with anti-CD3 (FITC anti-mouse CD3, Biolegend, 100204), anti-CD8 (PE anti-mouse CD8a, Biolegend, 100708) after staining with Fc-block. After fixation and permeabilization with commercial buffers (BD Cytofix/Cytoperm™ Fixation/Permeabilization Kit, 554714), cells were stained with anti-IFNγ antibody (PerCP/Cyanine5.5 anti-mouse IFN-γ, Biolegend, 505822). After routine washing and filtration, the cells were analyzed by flow cytometry.

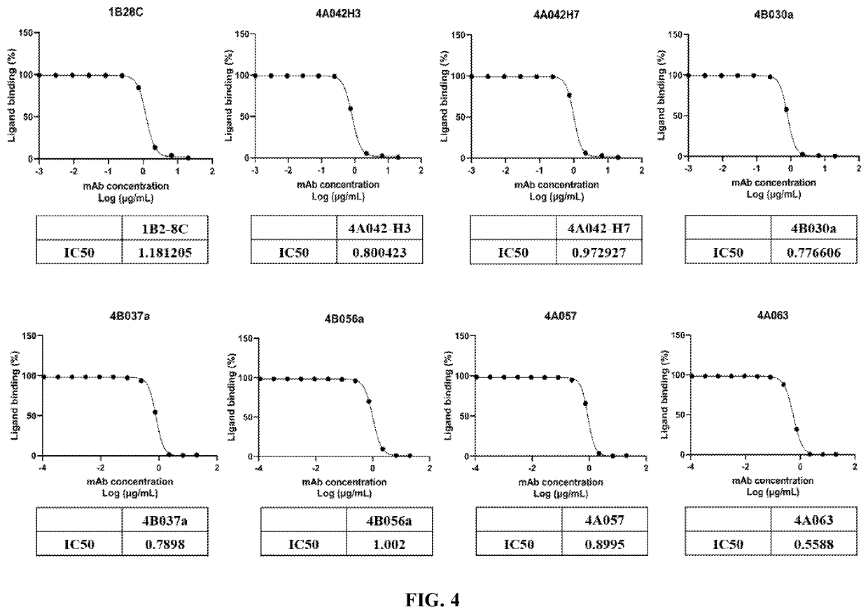

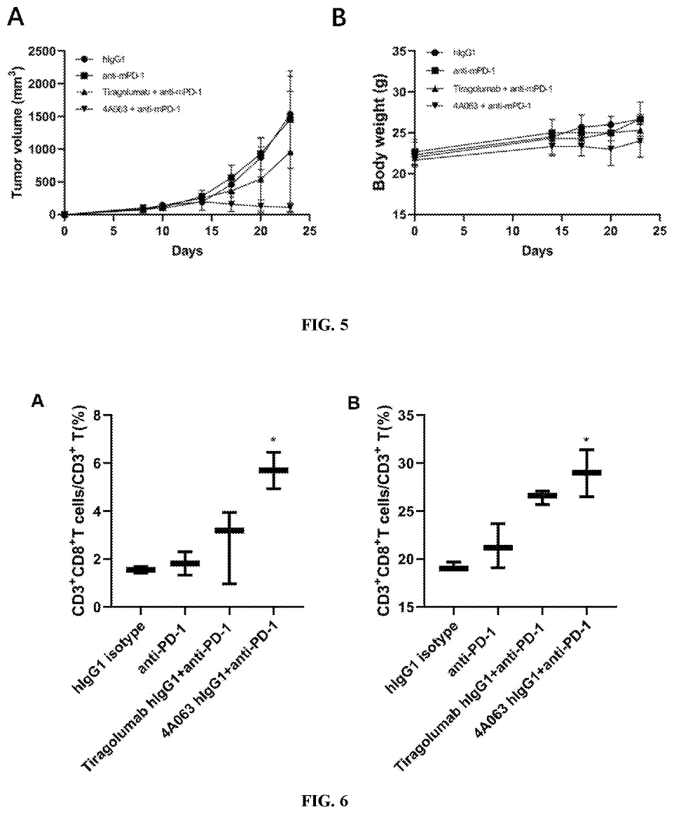

The results are shown in FIG. 6. FIG. 6A shows that in vivo treatment of tumor with anti-TIGIT antibody 4A063 hIgG1 results in an increase in proportion of CD8+TIL population in tumor microenvironment of combination treatment compared to the control group (P=0.0335), which is not observed in single PD-1 antibody group. At the same time, the flow cytometry results (which shown in FIG. 6B) of T cells in splenocytes were consistent with those in tumor microenvironment, which also showed that The proportion of CD8+ TIL population in the combination group was significantly higher than that in the subtype control group (P=0.0025), and also higher than that in the single PD-1 antibody treatment group (P=0.0171). This indicates the activation of cytotoxicity effector T cells after combination treatment, which may explain the difference between the combination and single PD-1 groups as discussed in Example 5. In the comparison within the combination treatment group, compared with the anti-TIGIT reference antibody (Tiragolumab-hIgG1) combination group, the increase of CD8+ TIL in 4A063-hIgG1 combination group was more obvious. For the PD-1 antibody combined with anti-TIGIT antibody 4A063 hIgG1 treatment group, the function of intratumoral T cells was also improved, and CD3+CD8+ T cells produce IFNγ increased.

All publications and patents cited in the present application are incorporated herein by reference. Without departing from the scope and spirit of the present invention, various modifications and variations of the described methods and compositions of the present invention will be apparent to those skilled in the art. Although the present invention has been described in terms of specific preferred embodiments, it should be understood that the claimed invention should not be unduly limited to these specific embodiments. In fact, many variations of the described modes for implementing the present invention that are obvious to those skilled in the relevant art are intended to be included within the scope of the claims.

1-21. (canceled)

22. An anti-TIGIT antibody or antigen-binding fragment thereof which binds to TIGIT (T cell immunoreceptor with Ig and ITIM domains),
wherein the anti-TIGIT antibody or antigen-binding fragment thereof comprises a heavy chain variable region (VH) comprising a CDRH1, a CDRH2, and a CDRH3, and/or a light chain variable region (VL) comprising a CDRL1, a CDRL2, and a CDRL3, wherein
(a) the CDRH1 comprises an amino acid sequence selected from the group consisting of GDSVSSNSAA (SEQ ID NO: 23) and GDSVSSNSAV (SEQ ID NO: 35);
(b) the CDRH2 comprises an amino acid sequence selected from the group consisting of TYYRSKWYN (SEQ ID NO: 24) and TYFRSKWYN (SEQ ID NO: 30);
(c) the CDRH3 comprises an amino acid sequence selected from the group consisting of AREGVLRYFDWLSGAFDI (SEQ ID NO: 25), ARERALYDILAGSFDY (SEQ ID NO: 31), and ARERGTYYDILTGDFDY (SEQ ID NO: 43);
(d) the CDRL1 comprises an amino acid sequence selected from the group consisting of QSVLYSSNNKNY (SEQ ID NO:20) and QSVLFGFNNKIY (SEQ ID NO: 26);
(e) the CDRL2 comprises an amino acid sequence of WAS; and
(f) the CDRL3 comprises an amino acid sequence selected from the group consisting of QQYYSTPWT (SEQ ID NO: 22), QQYYSFPYT (SEQ ID NO: 28), and QQHYSFPYT (SEQ ID NO: 40).

23. The anti-TIGIT antibody or antigen-binding fragment thereof according to claim 22, wherein
(a) the CDRH1 comprises an amino acid sequence of GDSVSSNSAA (SEQ ID NO: 23), the CDRH2 comprises an amino acid sequence of TYYRSKWYN (SEQ ID NO: 24), and the CDRH3 comprises an amino acid sequence of AREGVLRYFDWLSGAFDI (SEQ ID NO: 25);
(b) the CDRH1 comprises an amino acid sequence of GDSVSSNSAA (SEQ ID NO: 23), the CDRH2 comprises an amino acid sequence of TYFRSKWYN (SEQ ID NO: 30), and the CDRH3 comprises an amino acid sequence of ARERALYDILAGSFDY (SEQ ID NO: 31);
(c) the CDRH1 comprises an amino acid sequence of GDSVSSNSAV (SEQ ID NO: 35), the CDRH2 comprises an amino acid sequence of TYYRSKWYN (SEQ ID NO: 24), and the CDRH3 comprises an amino acid sequence of ARERALYDILAGSFDY (SEQ ID NO: 31); or
(d) the CDRH1 comprises an amino acid sequence of GDSVSSNSAA (SEQ ID NO: 23), the CDRH2 comprises an amino acid sequence of TYYRSKWYN (SEQ ID NO: 24), and the CDRH3 comprises an amino acid sequence of ARERGTYYDILTGDFDY (SEQ ID NO: 43).

24. The anti-TIGIT antibody or antigen-binding fragment thereof according to claim 22, wherein
(a) the CDRL1 comprises an amino acid sequence of QSVLYSSNNKNY (SEQ ID NO: 20, the CDRL2 comprises an amino acid sequence of WAS, and the CDRL3 comprises an amino acid sequence of QQYYSTPWT (SEQ ID NO: 22);
(b) the CDRL1 comprises an amino acid sequence of QSVLFGFNNKIY (SEQ ID NO: 26, the CDRL2 comprises an amino acid sequence of WAS, and the CDRL3 comprises an amino acid sequence of QQYYSFPYT (SEQ ID NO: 28); or
(c) the CDRL1 comprises an amino acid sequence of QSVLYSSNNKNY (SEQ ID NO: 20, the CDRL2 comprises an amino acid sequence of WAS, and the CDRL3 comprises an amino acid sequence of QQHYSFPYT (SEQ ID NO: 40).

25. The anti-TIGIT antibody or antigen-binding fragment thereof according to claim 22, wherein
(a) the CDRH1 comprises an amino acid sequence of GDSVSSNSAA (SEQ ID NO: 23), the CDRH2 comprises an amino acid sequence of TYYRSKWYN (SEQ ID NO: 24), the CDRH3 comprises an amino acid sequence of AREGVLRYFDWLSGAFDI (SEQ ID NO: 25), the CDRL1 comprises an amino acid sequence of QSVLYSSNNKNY (SEQ ID NO: 20), the CDRL2 comprises an amino acid sequence of WAS, and the CDRL3 comprises an amino acid sequence of QQYYSTPWT (SEQ ID NO: 22);
(b) the CDRH1 comprises an amino acid sequence of GDSVSSNSAA (SEQ ID NO: 23), the CDRH2 comprises an amino acid sequence of TYFRSKWYN (SEQ ID NO: 30), the CDRH3 comprises an amino acid sequence of ARERALYDILAGSFDY (SEQ ID NO: 31), the CDRL1 comprises an amino acid sequence of QSVLFGFNNKIY (SEQ ID NO: 26), the CDRL2 comprises an amino acid sequence of WAS, and the CDRL3 comprises an amino acid sequence of QQYYSFPYT (SEQ ID NO: 28);
(c) the CDRH1 comprises an amino acid sequence of GDSVSSNSAV (SEQ ID NO: 35), the CDRH2 comprises an amino acid sequence of TYYRSKWYN (SEQ ID NO: 24), the CDRH3 comprises an amino acid sequence of ARERALYDILAGSFDY (SEQ ID NO: 31), the CDRL1 comprises an amino acid sequence of QSVLFGFNNKIY (SEQ ID NO: 26), the CDRL2 comprises an amino acid sequence of WAS, and the CDRL3 comprises an amino acid sequence of QQYYSFPYT (SEQ ID NO: 28); or
(d) the CDRH1 comprises an amino acid sequence of GDSVSSNSAA (SEQ ID NO: 23), the CDRH2 comprises an amino acid sequence of TYYRSKWYN (SEQ ID NO: 24), the CDRH3 comprises an amino acid sequence of ARERGTYYDILTGDFDY (SEQ ID NO: 43), the CDRL1 comprises an amino acid sequence of QSVLYSSNNKNY (SEQ ID NO: 20), the CDRL2 comprises an amino acid sequence of WAS, and the CDRL3 comprises an amino acid sequence of QQHYSFPYT (SEQ ID NO: 40).

26. The anti-TIGIT antibody or antigen-binding fragment thereof according to claim 22, wherein the heavy chain variable region comprises an amino acid sequence selected from the group consisting of SEQ ID NO: 5, SEQ ID NO: 7, SEQ ID NO: 9, and SEQ ID NO: 11.

27. The anti-TIGIT antibody or antigen-binding fragment thereof according to claim 22, wherein the light chain variable region comprises an amino acid sequence selected from the group consisting of SEQ ID NO: 4, SEQ ID NO: 6, SEQ ID NO: 8, and SEQ ID NO: 10.

28. The anti-TIGIT antibody or antigen-binding fragment thereof according to claim 22, wherein
(a) the heavy chain variable region comprises an amino acid sequence of SEQ ID NO: 5 and the light chain variable region comprises an amino acid sequence of SEQ ID NO: 4;
(b) the heavy chain variable region comprises an amino acid sequence of SEQ ID NO: 7 and the light chain variable region comprises an amino acid sequence of SEQ ID NO: 6;
(c) the heavy chain variable region comprises an amino acid sequence of SEQ ID NO: 9 and the light chain variable region comprises an amino acid sequence of SEQ ID NO: 8; or
(d) the heavy chain variable region comprises an amino acid sequence of SEQ ID NO: 11 and the light chain variable region comprises an amino acid sequence of SEQ ID NO: 10.

29. An anti-TIGIT antibody or antigen-binding fragment thereof which binds to TIGIT (T cell immunoreceptor with Ig and ITIM domains),
wherein the anti-TIGIT antibody or antigen-binding fragment thereof comprises a heavy chain variable region (VH) comprising a CDRH1, a CDRH2, and a CDRH3, and/or a light chain variable region (VL) comprising a CDRL1, a CDRL2, and a CDRL3, wherein
(a) the CDRH1 comprises an amino acid sequence selected from the group consisting of GFSFDDYA (SEQ ID NO: 47), GFTFDDYA (SEQ ID NO: 53), GYTLTSHA (SEQ ID NO:59), GFNFDDYG (SEQ ID NO: 65), and GFTFDDFA (SEQ ID NO: 75);
(b) the CDRH2 comprises an amino acid sequence selected from the group consisting of ISWNSVTI (SEQ ID NO: 48), ISWNGIGI (SEQ ID NO: 54), INTNTGNP (SEQ ID NO: 60), and ISWNSISI (SEQ ID NO: 66);
(c) the CDRH3 comprises an amino acid sequence selected from the group consisting of AKELPTYRYYALDG (SEQ ID NO: 49), AKDGGDYHYYGMDV (SEQ ID NO: 55), ARGYSSPPRMDV (SEQ ID NO: 61), AKDGGDHYYYGMDV (SEQ ID NO: 67) and AKDKGLRRYYGMDV (SEQ ID NO: 77);
(d) the CDRL1 comprises an amino acid sequence selected from the group consisting of QSLLHSNGYNY (SEQ ID NO:44), QSLLHSNGYKY (SEQ ID NO: 50), QSVSRNY (SEQ ID NO: 56), and RSLLHSNGYNY (SEQ ID NO: 78);
(e) the CDRL2 comprises an amino acid sequence of LGS or GAS; and
(f) the CDRL3 comprises an amino acid sequence selected from the group consisting of MQALQTPLT (SEQ ID NO: 46), QQYGSSPFT (SEQ ID NO: 58), and MQALQTPPT (SEQ ID NO: 74).

30. The anti-TIGIT antibody or antigen-binding fragment thereof according to claim 22, further comprising a heavy chain constant region selected from a heavy chain constant region of IgG1, IgG2, IgG3, IgG4, IgA, IgM, IgE, and IgD.

31. The anti-TIGIT antibody or antigen-binding fragment thereof according to claim 30, wherein the heavy chain constant region selected comprises a heavy chain constant region of human IgG.

32. The anti-TIGIT antibody or antigen-binding fragment thereof according to claim 22, wherein the anti-TIGIT antibody or antigen-binding fragment thereof is in a form selected from the group consisting of F(ab′)₂, Fab′, Fab, Fv, scFv, bispecific antibody, and a combination thereof.

33. The anti-TIGIT antibody or antigen-binding fragment thereof according to claim 22, wherein the anti-TIGIT antibody or antigen-binding fragment thereof is conjugated to an agent.

34. The anti-TIGIT antibody or antigen-binding fragment thereof according to claim 33, wherein the agent is a detectable label or a cytotoxic agent.

35. A polynucleotide encoding the anti-TIGIT antibody or antigen-binding fragment thereof according to claim 22.

36. An expression vector enabling the expression of the anti-TIGIT antibody or antigen-binding fragment thereof according to claim 22.

37. An engineered cell comprising the polynucleotide according to claim 36.

38. A pharmaceutical composition comprising the anti-TIGIT antibody or antigen-binding fragment thereof according to claim 22, and a pharmaceutically acceptable carrier.

39. The pharmaceutical composition according to claim 38, further comprising another immune checkpoint inhibitor.

40. A method of treating a TIGIT-related disease comprising administering to a subject in need thereof an effective amount of the anti-TIGIT antibody or antigen-binding fragment thereof according to claim 22.


SEQUENCE LISTING (83 sequences)

SEQ ID NO: 1 - DNA, 735 bp, synthetic construct
  misc_feature 1..735: note = cDNA of human TIGIT
  source 1..735: mol_type = other DNA
atgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggttag

SEQ ID NO: 2 - protein, 120 aa, Homo sapiens
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 3 - DNA, 738 bp, synthetic construct
  misc_feature 1..738: note = cDNA of cynomolgus monkey TIGIT
  source 1..738: mol_type = other DNA
atgcggtggtgtctcttcctgatctgggcccaggggctgaggcaggctcccctcgcctcaggaatgatgacaggcacaatagaaacaacggggaacatttctgcaaagaaaggtggctctgttatcttacaatgtcacctctcctccaccatggcacaagtgacccaggtcaactgggagcagcatgaccattcgcttctggccattcgtaatgctgagttggggtggcacatctacccagccttcaaggatcgagtggccccgggtcctggcctgggcctcaccctccagtcgctgaccatgaatgatacaggggagtacttctgcacctatcacacctaccctgatgggacttacagagggagaatcttcctggaggtcctagaaagctcagtggctgagcacagtgccaggttccagattccattgcttggagccatggccatgatgctggtggtcatctgcatagcagtcatcgtggtggtcgtgttggctagaaagaagaaatccctcagaatccattctgtggaaagtggcctccagagaaaatcaactggacaggaagaacagattcccagtgctccctcacccccaggaagctgtgtccaggcagaagctgcacctgctgggctctgtggagagcagcagggagatgactgtgccgagctgcatgactacttcaatgtcctgagttacagaagcctggggagctgcagcttcttcacagagactgggtag

SEQ ID NO: 4 - protein, 113 aa, synthetic construct
  REGION 1..113: note = Antibody JHH1B2-8C VL
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPWTFGQGTKVEIK

SEQ ID NO: 5 - protein, 128 aa, synthetic construct
  REGION 1..128: note = Antibody JHH1B2-8C VH
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREGVLRYFDWLSGAFDIWGQGTMVTVSS

SEQ ID NO: 6 - protein, 113 aa, synthetic construct
  REGION 1..113: note = Antibody JHH4A042-H3 VL
DIVMTQSPDSLAVSLGERATINCKSSQSVLFGFNNKIYLAWYQQKPGQPPELLIYWASTRESGVPDRFSGSGSGTDFTLTISSLQAEDVAVYYCQQYYSFPYTFGQGTKLEIK

SEQ ID NO: 7 - protein, 126 aa, synthetic construct
  REGION 1..126: note = Antibody JHH4A042-H3 VH
QVQLQQSGPGLVKPSQTLSLTCAISGDSVSSNSAAWNWIRQSPSRGLEWLGRTYFRSKWYNDYAVSVKSRITINPDTSKNQFSLQLNSMTPEDTAVYYCARERALYDILAGSFDYWGQGTLVTVSS

SEQ ID NO: 8 - protein, 113 aa, synthetic construct; identical to SEQ ID NO: 6
  REGION 1..113: note = Antibody JHH4A042-H7 VL

SEQ ID NO: 9 - protein, 126 aa, synthetic construct
  REGION 1..126: note = Antibody JHH4A042-H7 VH
QVQLQQSGPGLVKPSQTLSLTCAISGDSVSSNSAVWNWIRQSPSRGLEWLGRTYYRSKWYNDYAVSVKSRITINPDTSKNQFSLQLNSMTPEDTAVYYCARERALYDILAGSFDYWGQGTLVTVSS

SEQ ID NO: 10 - protein, 113 aa, synthetic construct
  REGION 1..113: note = Antibody JHH4B030a VL
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQHYSFPYTFGQGTTLEIK

SEQ ID NO: 11 - protein, 127 aa, synthetic construct
  REGION 1..127: note = Antibody JHH4B030a VH
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARERGTYYDILTGDFDYWGQGTLVTVSS

SEQ ID NO: 12 - protein, 112 aa, synthetic construct
  REGION 1..112: note = Antibody JHH4B037a VL
DIVMTQSPLSLPVTPGEPASISCRSSQSLLHSNGYNYLDWFLQKPGQSPQLLIYLGSNRASGVPDRFSGSGSGTDFTLKISRVGAEDVGVYYCMQALQTPLTFGGGTKVEIK

SEQ ID NO: 13 - protein, 121 aa, synthetic construct
  REGION 1..121: note = Antibody JHH4B037a VH
EVQLVESGGGLVQPGRSLRLSCEASGFSFDDYAMHWVRQAPGKGLEWVSGISWNSVTIGYADSVKGRFTISRDNAKNSLYLEMNSLRTEDTALYYCAKELPTYRYYALDGWGQGTAVTVSS

SEQ ID NO: 14 - protein, 112 aa, synthetic construct
  REGION 1..112: note = Antibody JHH4B056a VL
DIVMTQSPLSLPVTPGEPASISCRSSQSLLHSNGYKYLNWYLQKPGQSPQLLIYLGSNRASGVPDRFSGSGSGTDFTLKISRVEAEDVGVYYCMQALQTPLTFGGGTKVEIK

SEQ ID NO: 15 - protein, 121 aa, synthetic construct
  REGION 1..121: note = Antibody JHH4B056a VH
EVQLVESGGGLVQPGRSLRLSCAASGFTFDDYAMHWVRQAPGKGLEWVSGISWNGIGIGYADSVKGRFTISRDNAKNSLYLQMNSLRAEDTALYYCAKDGGDYHYYGMDVWGQGTTVTVSS

SEQ ID NO: 16 - protein, 108 aa, synthetic construct
  REGION 1..108: note = Antibody JHH4A057 VL
EIVLTQSPGTLFLSPGERATLSCRASQSVSRNYLVWYQQKPGQAPRLLIYGASTRATGIPDRFSGSGSGTDFTLTIRRLEPEDFALYYCQQYGSSPFTFGQGTKLEIN

SEQ ID NO: 17 - protein, 119 aa, synthetic construct
  REGION 1..119: note = Antibody JHH4A057 VH
QVQLVQSGSELKKPGASVKVSCKASGYTLTSHALNWVRQAPGQGLEWMGWINTNTGNPTYVQGFTGRFVFSLDTSVSTAYLQISSLKAEDTAVYYCARGYSSPPRMDVWGQGTTVTVSS

SEQ ID NO: 18 - protein, 112 aa, synthetic construct
  REGION 1..112: note = Antibody JHH4A063 VL
DIMMTQSPLSLPVTPGEPASISCRSSQSLLHSNGYKYLNWYLQKPGQSPLLLIYLGSNRASGVPDRFSGSGSGTDFTLKISRVEAEDVGVYYCMQALQTPLTFGGGTKVEIK

SEQ ID NO: 19 - protein, 121 aa, synthetic construct
  REGION 1..121: note = Antibody JHH4A063 VH
EVQLVESGGGLVQPGRSLRLSCTASGFNFDDYGMHWVRQAPGKGLEWVSGISWNSISIGSADSVKGRFTISRDNAKNSLYLQMNSLRPEDTALYYCAKDGGDHYYYGMDVWGQGTTVTVSS

SEQ ID NO: 20 - protein, 12 aa, synthetic construct
  REGION 1..12: note = Antibody JHH1B2-8C VL CDR1
QSVLYSSNNKNY

SEQ ID NO: 21 - skipped

SEQ ID NO: 22 - protein, 9 aa, synthetic construct
  REGION 1..9: note = Antibody JHH1B2-8C VL CDR3
QQYYSTPWT

SEQ ID NO: 23 - protein, 10 aa, synthetic construct
  REGION 1..10: note = Antibody JHH1B2-8C VH CDR1
GDSVSSNSAA

SEQ ID NO: 24 - protein, 9 aa, synthetic construct
  REGION 1..9: note = Antibody JHH1B2-8C VH CDR2
TYYRSKWYN

SEQ ID NO: 25 - protein, 18 aa, synthetic construct
  REGION 1..18: note = Antibody JHH1B2-8C VH CDR3
AREGVLRYFDWLSGAFDI

SEQ ID NO: 26 - protein, 12 aa, synthetic construct
  REGION 1..12: note = Antibody JHH4A042-H3 VL CDR1
QSVLFGFNNKIY

SEQ ID NO: 27 - skipped

SEQ ID NO: 28 - protein, 9 aa, synthetic construct
  REGION 1..9: note = Antibody JHH4A042-H3 VL CDR3
QQYYSFPYT

SEQ ID NO: 29 - protein, 10 aa, synthetic construct
  REGION 1..10: note = Antibody JHH4A042-H3 VH CDR1
GDSVSSNSAA

SEQ ID NO: 30 - protein, 9 aa, synthetic construct
  REGION 1..9: note = Antibody JHH4A042-H3 VH CDR2
TYFRSKWYN

SEQ ID NO: 31 - protein, 16 aa, synthetic construct
  REGION 1..16: note = Antibody JHH4A042-H3 VH CDR3
ARERALYDILAGSFDY

SEQ ID NO: 32 - protein, 12 aa, synthetic construct
  REGION 1..12: note = Antibody JHH4A042-H7 VL CDR1
QSVLFGFNNKIY

SEQ ID NO: 33 - skipped

SEQ ID NO: 34 - protein, 9 aa, synthetic construct
  REGION 1..9: note = Antibody JHH4A042-H7 VL CDR3
QQYYSFPYT

SEQ ID NO: 35 - protein, 10 aa, synthetic construct
  REGION 1..10: note = Antibody JHH4A042-H7 VH CDR1
GDSVSSNSAV

SEQ ID NO: 36 - protein, 9 aa, synthetic construct
  REGION 1..9: note = Antibody JHH4A042-H7 VH CDR2
TYYRSKWYN

SEQ ID NO: 37 - protein, 16 aa, synthetic construct
  REGION 1..16: note = Antibody JHH4A042-H7 VH CDR3
ARERALYDILAGSFDY

SEQ ID NO: 38 - protein, 12 aa, synthetic construct
  REGION 1..12: note = Antibody JHH4B030a VL CDR1
QSVLYSSNNKNY

SEQ ID NO: 39 - skipped

SEQ ID NO: 40 - protein, 9 aa, synthetic construct
  REGION 1..9: note = Antibody JHH4B030a VL CDR3
QQHYSFPYT

SEQ ID NO: 41 - protein, 10 aa, synthetic construct
  REGION 1..10: note = Antibody JHH4B030a VH CDR1
GDSVSSNSAA

SEQ ID NO: 42 - protein, 9 aa, synthetic construct
  REGION 1..9: note = Antibody JHH4B030a VH CDR2
TYYRSKWYN

SEQ ID NO: 43 - protein, 17 aa, synthetic construct
  REGION 1..17: note = Antibody JHH4B030a VH CDR3
ARERGTYYDILTGDFDY

SEQ ID NO: 44 - protein, 11 aa, synthetic construct
  REGION 1..11: note = Antibody JHH4B037a VL CDR1
QSLLHSNGYNY

SEQ ID NO: 45 - skipped

SEQ ID NO: 46 - protein, 9 aa, synthetic construct
  REGION 1..9: note = Antibody JHH4B037a VL CDR3
MQALQTPLT

SEQ ID NO: 47 - protein, 8 aa, synthetic construct
  REGION 1..8: note = Antibody JHH4B037a VH CDR1
GFSFDDYA

SEQ ID NO: 48 - protein, 8 aa, synthetic construct
  REGION 1..8: note = Antibody JHH4B037a VH CDR2
ISWNSVTI

SEQ ID NO: 49 - protein, 14 aa, synthetic construct
  REGION 1..14: note = Antibody JHH4B037a VH CDR3
AKELPTYRYYALDG

SEQ ID NO: 50 - protein, 11 aa, synthetic construct
  REGION 1..11: note = Antibody JHH4B056a VL CDR1
QSLLHSNGYKY

SEQ ID NO: 51 - skipped

SEQ ID NO: 52 - protein, 9 aa, synthetic construct
  REGION 1..9: note = Antibody JHH4B056a VL CDR3
MQALQTPLT

SEQ ID NO: 53 - protein, 8 aa, synthetic construct
  REGION 1..8: note = Antibody JHH4B056a VH CDR1
GFTFDDYA

SEQ ID NO: 54 - protein, 8 aa, synthetic construct
  REGION 1..8: note = Antibody JHH4B056a VH CDR2
ISWNGIGI

SEQ ID NO: 55 - protein, 14 aa, synthetic construct
  REGION 1..14: note = Antibody JHH4B056a VH CDR3
AKDGGDYHYYGMDV

SEQ ID NO: 56 - protein, 7 aa, synthetic construct
  REGION 1..7: note = Antibody JHH4A057 VL CDR1
QSVSRNY

SEQ ID NO: 57 - skipped

SEQ ID NO: 58 - protein, 9 aa, synthetic construct
  REGION 1..9: note = Antibody JHH4A057 VL CDR3
QQYGSSPFT

SEQ ID NO: 59 - protein, 8 aa, synthetic construct
  REGION 1..8: note = Antibody JHH4A057 VH CDR1
GYTLTSHA

SEQ ID NO: 60 - protein, 8 aa, synthetic construct
  REGION 1..8: note = Antibody JHH4A057 VH CDR2
INTNTGNP

SEQ ID NO: 61 - protein, 12 aa, synthetic construct
  REGION 1..12: note = Antibody JHH4A057 VH CDR3
ARGYSSPPRMDV

SEQ ID NO: 62 - protein, 11 aa, synthetic construct
  REGION 1..11: note = Antibody JHH4A063 VL CDR1
QSLLHSNGYKY

SEQ ID NO: 63 - skipped

SEQ ID NO: 64 - protein, 9 aa, synthetic construct
  REGION 1..9: note = Antibody JHH4A063 VL CDR3
MQALQTPLT

SEQ ID NO: 65 - protein, 8 aa, synthetic construct
  REGION 1..8: note = Antibody JHH4A063 VH CDR1
GFNFDDYG

SEQ ID NO: 66 - protein, 8 aa, synthetic construct
  REGION 1..8: note = Antibody JHH4A063 VL CDR2
ISWNSISI

SEQ ID NO: 67 - protein, 14 aa, synthetic construct
  REGION 1..14: note = Antibody JHH4A063 VL CDR3
AKDGGDHYYYGMDV

SEQ ID NO: 68 - protein, 112 aa, synthetic construct
  REGION 1..112: note = Antibody JHH4D035a VL
DIVMTQSPLSLPVTPGEPASISCRSSQSLLHSNGYNYLDWYLQKPGQSPQLLIYLGSNRASGVPDRFSGSGSGTDFTLKISRMEAEDVGVYYCMQALQTPPTFGGGTKVEIR

SEQ ID NO: 69 - protein, 121 aa, synthetic construct
  REGION 1..121: note = Antibody JHH4D035a VH
EVQLVESGGGLVQPGRSLRLSCAASGFTFDDFAMHWVRQAPGKGLEWVSGISWNGIGIGYADSVKGRFTISRDNAKNSLYLQMNSLRAEDTALYFCAKDKGLRRYYGMDVWGQGITVIVSS

SEQ ID NO: 70 - protein, 112 aa, synthetic construct
  REGION 1..112: note = Antibody JHH4E061a VL
EIVMTQSPLSLPVTPGEPASISCRSSRSLLHSNGYNYLDWYLQKPGQSPQLLIYLGSNRASGVPDRFSGSGSGTDFTLKINRVEAEDVGVYYCMQALQTPPTFGGGTKVEIK

SEQ ID NO: 71 - protein, 121 aa, synthetic construct
  REGION 1..121: note = Antibody JHH4E061a VH
EVQLVESGGGLIQPGRSLTLSCVASGFTFDDYAMHWVRQAPGKGLEWVSGISWNGIGIGYADSVKGRFTISRDNAKNSLILQMNSLRIEDTALYYCAKDKGLRRYYGMDVWGQGTTVTVSS

SEQ ID NO: 72 - protein, 11 aa, synthetic construct
  REGION 1..11: note = Antibody JHH4D035a VL CDR1
QSLLHSNGYNY

SEQ ID NO: 73 - skipped

SEQ ID NO: 74 - protein, 9 aa, synthetic construct
  REGION 1..9: note = Antibody JHH4D035a VL CDR3
MQALQTPPT

SEQ ID NO: 75 - protein, 8 aa, synthetic construct
  REGION 1..8: note = Antibody JHH4D035a VH CDR1
GFTFDDFA

SEQ ID NO: 76 - protein, 8 aa, synthetic construct
  REGION 1..8: note = Antibody JHH4D035a VL CDR2
ISWNGIGI

SEQ ID NO: 77 - protein, 14 aa, synthetic construct
  REGION 1..14: note = Antibody JHH4D035a VL CDR3
AKDKGLRRYYGMDV

SEQ ID NO: 78 - protein, 11 aa, synthetic construct
  REGION 1..11: note = Antibody JHH4E061a VL CDR1
RSLLHSNGYNY

SEQ ID NO: 79 - skipped

SEQ ID NO: 80 - protein, 9 aa, synthetic construct
  REGION 1..9: note = Antibody JHH4E061a VL CDR3
MQALQTPPT

SEQ ID NO: 81 - protein, 8 aa, synthetic construct
  REGION 1..8: note = Antibody JHH4E061a VH CDR1
GFTFDDYA

SEQ ID NO: 82 - protein, 8 aa, synthetic construct
  REGION 1..8: note = Antibody JHH4E061a VH CDR2
ISWNGIGI

SEQ ID NO: 83 - protein, 14 aa, synthetic construct
  REGION 1..14: note = Antibody JHH4E061a VH CDR3
AKDKGLRRYYGMDV